SEQUENCE LISTING (10 sequences)

SEQ ID NO: 1 - DNA, 2280 bp, Nicotiana tabacum
  source 1..2280: mol_type = other DNA; note = Polynucleotide coding sequence of LKR-S from Nicotiana tabacum
  CDS 1..2280: protein_id = 2; translation = MTDLLKFGREILGPVIMFGNGVVGILSEATNKWERRAPLTPSHC ARLLHGGRGKTGVSRIIMQPSTKRVHHDALYEDVGCEISEDLSDCGLILGIKQPKLEM ILPDRAYAFFSHTHKAQKENMPLLDKILAERASLFDYELIVGDTGKRLLAFGSFAGRA GMIDFLRGLGLWYLNHGYSTPFLSLGSSYMYSSLAAAKAAVISVGEEIATMGLPAGIC PLVFVFTGSGNVSRGAQEIFKLLPHTFVDPRKLSELHETARDLTQSKHPSKRIFQVYG CVTTCQDMVEHLNPSKSFDKIDYYAHPEQYRPAFHEKIAPYVSVIVNCMYWEKRFPRL LTTKQIQDLMRNGCPLVGICDITCDVGGSIEFINQTSSIDSPFFRYEPSNDSYHYDIE GKGVMCSAVDILPTEFAKEASQHFGDILSHFTGSLASFRNLEELPAHLKRACIAHHGA LTQLYEYIPRMRKSDLEDPSTVLSSSNANGRKYTVLVSLSGHLFDKFLINEALDIIEA AGGSFHLVKCQVGQITSALSYSELEVGAEDKAVLDKIVDSLTSLANSRNSLGSQNKEN NMISLKVGEFQQSIIDEKSDAKKVLILGAGRVCRPAAELLASIGSMSSGQWLSSITAD FEEQHCVQVIVASLYLKDAEEVTEGIPNAKAVQLDIMNHESLSSCISQVDVVISLLPP SCHGIVAKSCIELKKHLVTASYVNDSMLKLDEDAKCAGITILGEMGLDPGIVTLIRVS MNLLENSSWYEISSECDCA
atgactgatttgctaaaatttggtcgagagattcttggtccagtcataatgtttggaaatggagtagttggtatcctctcagaggcgaccaacaaatgggagagacgagctcctctgactccttcacactgtgctcgattgctgcatggtgggaggggtaagactggagtgtcgcgcatcatcatgcagccatccacgaagcgtgttcatcatgatgctctctatgaggacgttggatgcgagatttctgaggacttgtccgactgtggtctcatattgggtatcaaacagcccaagttggagatgattcttcctgatagggcctatgcattcttctcgcatactcacaaggcacaaaaggagaatatgcccttgttggataagatattagctgaaagggcatctctgttcgactacgagcttatagttggagacactgggaagaggttacttgcatttggaagttttgctggtcgagctggaatgattgattttctacgcggattaggactgtggtatctcaaccatggctactcaacgccctttctctcactcggctcatcgtacatgtactcttcattggctgctgctaaagctgcagtaatttctgttggcgaagagatagcgaccatggggcttccggcgggaatatgtccccttgtatttgttttcactggttctggaaacgtgtctcgtggtgcacaagaaatctttaagcttcttcctcatacttttgtggatccaagaaaactttcagaactacatgagacagctagggatcttactcaatccaaacatccatcaaagagaatctttcaagtgtatggttgcgtaacgacctgccaagacatggtggaacatttgaatccttcaaaatcctttgataagattgattactatgcacatccagagcaatacagacctgctttccatgagaaaatagccccttatgtatctgtcatagtaaattgtatgtactgggagaagaggtttcctaggttgttgactactaagcagattcaagacctcatgagaaatggatgcccacttgttggaatctgcgacataacatgtgatgtgggaggctcaatcgagttcatcaaccaaacatcatcaatagactcgccttttttcaggtacgaaccttccaatgattcatatcattatgacatagaaggtaaaggtgtgatgtgttcagctgttgatattcttcccaccgagtttgcaaaagaggcatcccaacatttcggagatattttgtcccactttacgggaagcttagcttcctttagaaatcttgaggagttacctgcacacttaaagagagcttgcatagcccatcacggagctcttactcagttgtatgaatacatccctcgaatgcggaaatctgacctagaagacccttcgacagttctttctagttccaatgcaaacgggaggaaatacactgttttggtatctctgagtggtcatttgtttgataaattcctcataaacgaagctttggatattattgaagcagcaggtggctccttccacttggtgaagtgccaagttggtcaaatcacaagcgctttgtcatactcagaactggaggttggagctgaagataaagcagttttagataaaatagttgattctttaacttcccttgctaattcaagaaattctttgggatctcaaaacaaggaaaacaacatgatatcgttgaaggttggggaattccaacaaagcatcatagatgagaaatctgatgcaaagaaggtcttaattttaggtgctggtcgggtttgcagaccagctgcagaacttttagcatcaataggtagcatgtcatccggacaatggttatcatctataactgctgattttgaagagcaacattgtgttcaagtaattgttgcttctttgtacttgaaggatgcagaagaggttactgaaggcattccaaatgcaaaagcagttcaacttgatattatgaatcatgagtctctttctagttgcatctcacaggttgatgttgtcatcagcttactgcctcctagttgccatggtattgtagcaaaatcatgcattgagctgaagaaacatcttgtcacagctagctacgttaatgattctatgttaaagctagacgaagacgcaaaatgtgctggaattactattcttggtgaaatgggcttggacccaggaatagtaactctaataagagtgtcaatgaatttgttggaaaattcttcatggtatgaaatttcctctgaatgtgattgtgcataa

SEQ ID NO: 2 - protein, 759 aa, Nicotiana tabacum
  source 1..759: note = Polypeptide sequence of LKR-S from Nicotiana tabacum
MTDLLKFGREILGPVIMFGNGVVGILSEATNKWERRAPLTPSHCARLLHGGRGKTGVSRIIMQPSTKRVHHDALYEDVGCEISEDLSDCGLILGIKQPKLEMILPDRAYAFFSHTHKAQKENMPLLDKILAERASLFDYELIVGDTGKRLLAFGSFAGRAGMIDFLRGLGLWYLNHGYSTPFLSLGSSYMYSSLAAAKAAVISVGEEIATMGLPAGICPLVFVFTGSGNVSRGAQEIFKLLPHTFVDPRKLSELHETARDLTQSKHPSKRIFQVYGCVTTCQDMVEHLNPSKSFDKIDYYAHPEQYRPAFHEKIAPYVSVIVNCMYWEKRFPRLLTTKQIQDLMRNGCPLVGICDITCDVGGSIEFINQTSSIDSPFFRYEPSNDSYHYDIEGKGVMCSAVDILPTEFAKEASQHFGDILSHFTGSLASFRNLEELPAHLKRACIAHHGALTQLYEYIPRMRKSDLEDPSTVLSSSNANGRKYTVLVSLSGHLFDKFLINEALDIIEAAGGSFHLVKCQVGQITSALSYSELEVGAEDKAVLDKIVDSLTSLANSRNSLGSQNKENNMISLKVGEFQQSIIDEKSDAKKVLILGAGRVCRPAAELLASIGSMSSGQWLSSITADFEEQHCVQVIVASLYLKDAEEVTEGIPNAKAVQLDIMNHESLSSCISQVDVVISLLPPSCHGIVAKSCIELKKHLVTASYVNDSMLKLDEDAKCAGITILGEMGLDPGIVTLIRVSMNLLENSSWYEISSECDCA

SEQ ID NO: 3 - DNA, 3114 bp, Nicotiana tabacum
  source 1..3114: mol_type = other DNA; note = Polynucleotide coding sequence of LKR-T from Nicotiana tabacum
  CDS 1..3114: protein_id = 4; translation = MFGNGVVGILSEATNKWERRAPLTPSHCARLLHTGVSRIIVQPS TKRVHHDALYEDVGCEISEDLSDCGLILGIKQPKLEMILPDRAYAFFSHTHKAQKENM PLLDMILAERASLFDYELIVGDTGKRLLAFGSFAGRAGMIDFLRGLGLWYLNHGYSTP FLSLGSSYMYSSLAAAKAAVISVGEEIATMGLPAGICPLVFVFTGAGNVSRGAQEIFK LLPHTFVDPRKLSELHETARDLTQSKHPSKRIFQVYGCVTTCQDMVERLNPSKSFDKI DYYAHPEQYGPAFHEKIAPYASVIVNCMYWEKRFPRLLTTKQIQDLMRNGCPLVGICD ITCDVGGSIEFINQTSSIDSPFFRYEPSNDSYHFDIEGKGVMCSAVDILPTEFAKEAS QHFGDILSHFTGSLASFRNLEERACIAHHGRLTQLYEYIPRMRKSDLEDPSTVLSSSN ANGRKYTVLVSLSGHLFDKFLINEALDIIEAAGGSFHLVKCQVGQITSALSYSELEVG AEDKAVLDKIVDSLTSLAYSRNSLGSQNKENNMISLKVGEFQQSITDEKSDAKVLILG AGRVCRPAAELLASIGSMSSGQCLSSITADFEEQNCVQVIVASLYLKDAEEVTEGIPN AKAVQLDITNHESLSSCISQVDVVISLLPPSCHGIVAKSCIELKKHLVTASYVNDSML KLDEDAKCAGITILGEMGLDPGIDHMMAMKMINQAHAANGKIRSFVSYCGGLPSPAAA NNPLAYKFSWNPAGAIRAGWNPAAYRSEGEIIHVEGQRLYDSAAKLRLPDFPAFALEC LPNRNSLVYGDLYGVGEEASTIFRGTLRYEGFSQIMGTLAKIGFFCTEPSLILKDGIK PTHRAFLLGLVGIDGQIFPEPVIDEKYITDRILKLGLCKDKDTAVKTAKTIIFLGFQE PTEIPSSCKSPFEVTCLRMEERLAYSNTEQDMVLLHHEVVVDYPDGHAETHRSTLLEM GRTANGKTNMAMSLTVGIPAATGALLLLANKIKANGVLRPIDPEVYEPALDILEAYGF KLLEKIE
atgtttggaaatggtgtagttggtatcctctcagaggcgacgaacaagtgggaaagacgagctcctctgactccttcacattgtgctcgattgctgcatactggagtgtcgcgcatcatcgtgcagccatccacaaagcgtgttcatcatgatgctctctatgaggatgttggatgcgagatttctgaggacttgtccgactgtggtctcatattgggtatcaaacagcccaagttggagatgattcttcctgatagggcctatgcattcttctcacatactcacaaggcacaaaaggaaaatatgcccttgttggatatgatactagctgaaagggcttctctgttcgactatgagcttatagttggagacactgggaagaggttacttgcgtttggaagttttgctggtcgagctggaatgattgattttctacgcggattaggactgtggtatctcaaccatggctactcaacgccctttctctcactcggctcatcgtacatgtactcttcattggctgctgctaaggctgcagtaatttctgttggcgaagagatagcgaccatggggcttccagcgggaatatgtcctcttgtatttgttttcactggtgctggaaatgtgtctcgtggtgcacaagaaatttttaagcttcttcctcatacatttgtggatccaagaaaactttcagaactacatgagacggctagggatcttactcaatccaaacatccatcaaagagaatctttcaagtgtatggttgcgtaacgacctgccaagacatggtggaacgtttaaatccttcaaaatcctttgataagattgattactatgcacatccagagcaatacggacctgctttccatgagaaaatagccccttatgcatctgtcatagtaaattgtatgtactgggagaagaggtttcctaggttgttgactactaagcagattcaagacctcatgagaaatggatgcccacttgttggaatctgtgacataacatgtgatgtgggaggctcgatcgaattcatcaaccaaacatcatcaatagactcgccttttttcaggtatgaaccttccaatgattcatatcattttgacatagaaggtaaaggtgtgatgtgttccgctgttgatattcttcccaccgagtttgcaaaagaggcatcccaacatttcggagatattttgtcccactttacgggaagcttagcttcctttagaaatcttgaggagagagcttgcatagcccatcacggacgtcttactcagttgtatgaatacatccctcgaatgcggaaatctgacctagaagacccttcaacagttctttctagttccaatgcaaacgggaggaaatacaccgttttggtatctctgagtggtcatttgtttgataaattcctcataaacgaagccttggatattattgaagcagcaggtggctccttccacttggtgaagtgccaagttggtcaaatcacaagcgctttgtcatactcagaactggaggttggagctgaagataaagcagttttggataaaatagttgattctttaacttcccttgcttattcaagaaattctttgggatctcaaaacaaggaaaacaacatgatatctttgaaggttggggaattccaacaaagcatcacagatgagaaatctgatgcaaaggtcttaattttaggtgctggtcgggtttgcagaccagctgcagaacttttagcatcaataggtagcatgtcatccggacaatgcttatcatctataactgctgattttgaagagcaaaattgtgttcaagtgattgttgcttctttgtacttgaaggatgcggaagaggttactgaaggtattccaaatgcaaaagcagttcagcttgatattacgaatcacgagtctctttctagttgcatctcacaggttgatgttgtcatcagcttactgcctcctagttgccatggtattgtagcaaaatcatgcattgagctgaagaaacatcttgtcacagctagctacgttaatgattcaatgttaaagctagacgaagacgcaaaatgtgctggaattactattcttggtgaaatgggcttggatccaggaatagatcatatgatggcaatgaaaatgatcaaccaagcccatgcagcaaacggaaagatcaggtctttcgtttcttactgtggtggtcttccctctccagctgcggccaacaacccactagcttataagttcagttggaacccagccggagctatacgagctgggtggaatccagcagcctatagatctgaaggggaaattattcatgttgaaggtcagaggctttatgattcagctgcaaagcttcgtcttcctgattttccagcttttgcattagagtgtctcccaaatcgcaactccttagtgtatggagacttatatggtgtaggagaggaagcatcaacaatctttcgaggaacactacgctatgaaggttttagtcaaataatggggacacttgccaagattggattcttctgtacagaaccatctcttattcttaaagatgggattaaacccacacacagagcatttttgctgggacttgttggaatagacggacagattttccccgaaccagtaattgatgaaaaatacatcactgataggatcttgaaacttgggctttgtaaagacaaggacactgcagttaagacagcaaaaactataatatttttgggatttcaagagcctacagaaataccatcatcctgcaaatctccatttgaagttacgtgcttgcgcatggaagagagattagcatactccaatacagagcaggacatggtgcttttacaccatgaagtggtagtagattacccagatggccatgctgaaacccatagatccacacttttggaaatggggaggacagcgaatgggaaaaccaacatggccatgtctcttacagtagggattcccgcagccactggagctctgctattacttgcgaacaagatcaaagcaaatggtgtactaaggcctattgatccagaagtttatgagccagcactggatattttggaggcatatggtttcaagttgctggagaagattgaataa

SEQ ID NO: 4 - protein, 1037 aa, Nicotiana tabacum
  source 1..1037: note = Polypeptide sequence of LKR-T from Nicotiana tabacum
MFGNGVVGILSEATNKWERRAPLTPSHCARLLHTGVSRIIVQPSTKRVHHDALYEDVGCEISEDLSDCGLILGIKQPKLEMILPDRAYAFFSHTHKAQKENMPLLDMILAERASLFDYELIVGDTGKRLLAFGSFAGRAGMIDFLRGLGLWYLNHGYSTPFLSLGSSYMYSSLAAAKAAVISVGEEIATMGLPAGICPLVFVFTGAGNVSRGAQEIFKLLPHTFVDPRKLSELHETARDLTQSKHPSKRIFQVYGCVTTCQDMVERLNPSKSFDKIDYYAHPEQYGPAFHEKIAPYASVIVNCMYWEKRFPRLLTTKQIQDLMRNGCPLVGICDITCDVGGSIEFINQTSSIDSPFFRYEPSNDSYHFDIEGKGVMCSAVDILPTEFAKEASQHFGDILSHFTGSLASFRNLEERACIAHHGRLTQLYEYIPRMRKSDLEDPSTVLSSSNANGRKYTVLVSLSGHLFDKFLINEALDIIEAAGGSFHLVKCQVGQITSALSYSELEVGAEDKAVLDKIVDSLTSLAYSRNSLGSQNKENNMISLKVGEFQQSITDEKSDAKVLILGAGRVCRPAAELLASIGSMSSGQCLSSITADFEEQNCVQVIVASLYLKDAEEVTEGIPNAKAVQLDITNHESLSSCISQVDVVISLLPPSCHGIVAKSCIELKKHLVTASYVNDSMLKLDEDAKCAGITILGEMGLDPGIDHMMAMKMINQAHAANGKIRSFVSYCGGLPSPAAANNPLAYKFSWNPAGAIRAGWNPAAYRSEGEIIHVEGQRLYDSAAKLRLPDFPAFALECLPNRNSLVYGDLYGVGEEASTIFRGTLRYEGFSQIMGTLAKIGFFCTEPSLILKDGIKPTHRAFLLGLVGIDGQIFPEPVIDEKYITDRILKLGLCKDKDTAVKTAKTIIFLGFQEPTEIPSSCKSPFEVTCLRMEERLAYSNTEQDMVLLHHEVVVDYPDGHAETHRSTLLEMGRTANGKTNMAMSLTVGIPAATGALLLLANKIKANGVLRPIDPEVYEPALDILEAYGFKLLEKIE

SEQ ID NO: 5 - DNA, 110 bp, synthetic construct
  source 1..110: mol_type = other DNA; note = NtLKR-RNAi insert
caggttgatgttgtcatcagcttactgcctcctagttgccatggtattgtagcaaaatcatgcattgagctgaagaaacatcttgtcacagctagctacgttaatgattc

SEQ ID NO: 6 - DNA, 23 bp, synthetic construct
  source 1..23: mol_type = other DNA; note = NtLKR-F1 primer for qPCR
atattattgaagcagcaggtggc

SEQ ID NO: 7 - DNA, 23 bp, synthetic construct
  source 1..23: mol_type = other DNA; note = NtLKR-R1 primer for qPCR
tgctttatcttcagctccaacct

SEQ ID NO: 8 - DNA, 110 bp, synthetic construct; identical to SEQ ID NO: 5
  source 1..110: mol_type = other DNA; note = DNA fragment used for suppressing the expression of NtLKR-S and NtLKR-T

SEQ ID NO: 9 - DNA, 22 bp, synthetic construct
  source 1..22: mol_type = other DNA; note = MMV-F Primer
gacgtctaatcccaacttcgtc

SEQ ID NO: 10 - DNA, 22 bp, synthetic construct
  source 1..22: mol_type = other DNA; note = IPMS2-R primer
gacgtctaatcccaacttcgtc

(19) United States
(12) Patent Application Publication
(10) Pub. No.: US 2026/0242812 A1
(43) Pub. Date: Aug. 20, 2026

(54) MODULATION OF GENES CODING FOR LYSINE KETOGLUTARATE REDUCTASE

(71) Applicant: PHILIP MORRIS PRODUCTS S.A., Neuchatel (CH)

(72) Inventors: Lucien Bovet, La Chaux-de-Fonds (CH); Aurore Ginette Denise Hilfiker, Salavaux (CH)

(21) Appl. No.: 19/152,474

(22) PCT Filed: Jan. 31, 2024
(86) PCT No.: PCT/EP2024/052296

§ 371 Date: Jul. 31, 2025

(30) Foreign Application Priority Data
Feb. 2, 2023 (EP) 23154681.3

(51) Int. Cl. C12N 15/82; C12N 9/06

(52) U.S. Cl. CPC C12N 15/8243; C12N 9/0028; C12N 15/8218; C12N 2310/14; C12Y 105/01008

(57) ABSTRACT

There is disclosed a mutant, non-naturally occurring or transgenic Nicotiana tabacum plant or part thereof having modulated expression or activity of lysine ketoglutarate reductase (LKR), said LKR comprising, consisting or consisting essentially of: (i) a polynucleotide(s) comprising, consisting or consisting essentially of a sequence having at least 88% sequence identity to the SEQ ID NO: 1 (NtLKR-S) and/or having at least 86% sequence identity to SEQ ID NO: 3 (NtLKR-T); (ii) a polypeptide(s) encoded by the polynucleotide(s) set forth in (i); (iii) a polypeptide(s) comprising, consisting or consisting essentially of a sequence having at least 89% sequence identity to SEQ ID NO: 2 (NtLKR-S) and/or having at least 88% sequence identity to SEQ ID NO: 4 (NtLKR-T); or (iv) a construct, vector or expression vector comprising the isolated polynucleotide(s) set forth in (i), wherein said plant or part thereof comprises at least one modification which is capable of modulating (a) the expression of the polynucleotide(s) in the plant or part thereof; or (b) the activity of the polypeptide(s) in the plant or part thereof, as compared to a control plant or part thereof in which the expression of the polynucleotide(s) or the activity of the polypeptide(s) has not been modified.

### FIELD OF THE INVENTION

The present invention relates in general to Nicotiana tabacum plants having modulated expression or activity of lysine ketoglutarate reductase (LKR) (also known as saccharopine dehydrogenase).

### BACKGROUND

To manufacture tobacco products, different types of tobaccos are mixed at various ratios to create blends with certain flavour characteristics. Flue-cured tobacco (for example, Virginia) is the most widely grown tobacco and is characterised by a high ratio of sugar to nitrogen but it has a limited flavour profile. Other tobacco types—such as air-cured (for example, Burley, Maryland and Galpao) or fire-cured (for example, Dark) tobacco types—offer alternative flavour profiles. These different flavour profiles are important in the production of blended tobacco products. The flavour characteristics are the result of particular flavour compounds or the precursors for these compounds that are present at certain levels in tobacco plants. Since the varieties of tobacco for commercial production are limited, this means that the opportunities to develop tobacco products with different flavour and aroma profiles are also limited. This equally applies to the manufacture of reconstituted tobacco material that is used in heated tobacco sticks in reduced risk products.

There remains a need in the art to improve the opportunities to create tobacco that offers new flavour and/or sensory experiences for consumers, whilst still retaining commercially acceptable yields and traits. The present invention seeks to address this and other needs.

### SUMMARY OF THE INVENTION

The present invention is based, at least in part, on the surprising finding that modulation of Nicotiana tabacum LKR polynucleotide expression or Nicotiana tabacum LKR polypeptide expression can alter the chemical profile of cured Nicotiana tabacum plant material—such as cured leaf. Advantageously, this can be achieved without generating an abnormal phenotype in the Nicotiana tabacum plants thereby conferring commercially acceptable yields and traits. Without wishing to be bound by theory, it is believed that changes in chemistry occur via changes in the senescence pathway (during early curing), which can modulate the levels of essential amino acids—such as lysine. Additional consistent changes in other amino acids (and also sugars etc) are observed such that is it possible to engineer tobacco materials with different flavour and/or sensory properties. This is of value for those types of tobacco that are widely grown commercially but have a limited flavour profile.

Zhu et al. (2001) Plant Physiol. 126(4):1539-45 described an Arabidopsis knockout mutant of LKR. The phenotype of the knockout was indistinguishable from wild-type plants under normal growth conditions. The relative level of free Lys in leaves was measured and found to be similar between the wild-type and LKR knockout mutant. No major differences were observed between leaves of the wild type and LKR knockout mutant in the relative levels of other free amino acids. It is surprising that, in contrast to the results seen in Arabidopsis, modulation of LKR can alter the amino acid profile of cured Nicotiana tabacum plant material. For example, it is unexpected that decreasing LKR expression or activity in Nicotiana tabacum can enrich the cured leaf with certain essential amino acids, as described herein.

There is disclosed a mutant, non-naturally occurring or transgenic or genetically engineered Nicotiana tabacum plant or part thereof (such as leaf, suitably cured leaf) having modulated (for example, increased or decreased, suitably decreased) expression or activity of LKR, said LKR comprising, consisting or consisting essentially of: (i) a polynucleotide(s) comprising, consisting or consisting essentially of a sequence having at least 88% sequence identity to the SEQ ID NO: 1 (NtLKR-S) and/or having at least 86% sequence identity to SEQ ID NO: 3 (NtLKR-T); (ii) a polypeptide(s) encoded by the polynucleotide set forth in (i); (iii) a polypeptide comprising, consisting or consisting essentially of a sequence having at least 89% sequence identity to SEQ ID NO: 2 (NtLKR-S) and/or having at least 88% sequence identity to SEQ ID NO: 4 (NtLKR-T); or (iv) a construct, vector or expression vector comprising the isolated polynucleotide(s) set forth in (i), wherein said plant or part thereof comprises at least one modification which is capable of modulating: (a) the expression of the polynucleotide(s) in the plant or part thereof; or (b) the activity of the polypeptide(s) in the plant or part thereof, as compared to a control plant or part thereof in which the expression of the polynucleotide(s) or the activity of the polypeptide(s) has not been modified.

Suitably, the modification comprises at least one genetic alteration in a coding sequence of the polynucleotide(s) or in a regulatory region of the polynucleotide(s).

Suitably, the modification comprises one or more of exogenous DNA or exogenous RNA.

Suitably, the modification comprises one or more of a vector or a viral vector or an Agrobacterium vector or a CRISPR vector.

Suitably, the modification is capable of driving one or more of RNA interference or transcriptional gene silencing or virus induced gene silencing.

Suitably, the modification is capable of expressing one or more of double stranded RNA (dsRNA) or hairpin RNA (hpRNA) or small interfering RNA.

Suitably, the modulated expression or activity of LKR confers a modulation in the level of one or more amino acids in the plant or part thereof as compared to the level of the one or more amino acids in the control plant, suitably, wherein the modulated expression or activity of LKR confers a modulation in the timing of leaf senescence.

Suitably, the amino acid is lysine.

Suitably, the part of the mutant, non-naturally occurring or transgenic Nicotiana tabacum plant is cured or dried leaf.

Suitably, the levels of at least lysine, arginine, GABA, glutamine, alanine, tyrosine, isoleucine and threonine are modulated in the cured or dried leaf as compared to cured or dried leaf from the control plant.

Suitably, the Nicotiana tabacum plant or part thereof is a Burley type.

Suitably, in the cured or dried leaf the levels of at least lysine, arginine, proline, GABA, glutamine, leucine, alanine, phenylalanine, tyrosine, isoleucine, methionine, threonine and glycine are modulated and there is no significant change in the levels of at least asparagine, aspartic acid, tryptophan, histidine, glutamic acid, serine, and valine as compared to cured or dried leaf from the control plant.

Suitably, in the cured or dried leaf the expression of the LKR polynucleotide or the activity of the LKR polypeptide is decreased or inhibited and wherein in the cured or dried leaf: (i) the levels of at least lysine, arginine, proline, GABA, glutamine, leucine, alanine, phenylalanine, tyrosine, and isoleucine are increased; and (ii) the levels of at least methionine, threonine and glycine are decreased; and (iii) there is no significant change in the levels of at least asparagine, aspartic acid, tryptophan, histidine, glutamic acid, serine and valine as compared to cured or dried leaf from the control plant.

Suitably, the Nicotiana tabacum plant or part thereof is a Virginia type.

Suitably, in the cured or dried leaf the levels of at least lysine, arginine, glutamine, histidine, tyrosine, tryptophan, threonine, GABA, asparagine, alanine, isoleucine, valine and serine are modulated and there is no significant change in the levels of at least proline, aspartic acid, leucine, phenylalanine, glutamic acid and methionine as compared to cured or dried leaf from the control plant.

Suitably, in the cured or dried leaf the expression of the LKR polynucleotide or the activity of the LKR polypeptide is decreased or inhibited and wherein in the cured or dried leaf: (i) the levels of at least lysine, arginine, glutamine, histidine, tyrosine, tryptophan, threonine, GABA, asparagine and alanine are increased; and (ii) the levels of at least isoleucine, valine and serine are decreased; and (iii) there is no significant change in the levels of at least proline, aspartic acid, leucine, phenylalanine, glutamic acid and methionine as compared to cured or dried leaf from the control plant.

Suitably, the sum of sugars is modulated, suitably decreased.

In a further aspect, there is disclosed a Nicotiana tabacum plant material, cured Nicotiana tabacum plant material, or homogenized Nicotiana tabacum plant material, derived or obtained from the Nicotiana tabacum plant or part thereof described above; suitably, wherein the Nicotiana tabacum plant material is selected from the group consisting of biomass, seed, stem, flower, or leaf or a combination of two or more thereof; suitably, wherein the Nicotiana tabacum plant material is leaf; suitably, wherein the leaf is cured leaf; suitably, wherein the cured leaf is selected from the group consisting of flue-cured leaf, sun-cured leaf or air-cured leaf.

In a further aspect, there is disclosed a method for producing a Nicotiana tabacum plant in which the level of at least one amino acid is modulated comprising: (a) providing a Nicotiana tabacum plant comprising: (i) a polynucleotide(s) comprising, consisting or consisting essentially of a sequence having at least 88% sequence identity to the SEQ ID NO: 1 (NtLKR-S) and/or having at least 86% sequence identity to SEQ ID NO: 3 (NtLKR-T); (ii) a polypeptide(s) encoded by the polynucleotide(s) set forth in (i); (iii) a polypeptide(s) comprising, consisting or consisting essentially of a sequence having at least 89% sequence identity to SEQ ID NO: 2 (NtLKR-S) and/or having at least 88% sequence identity to SEQ ID NO: 4 (NtLKR-T); or (iv) a construct, vector or expression vector comprising the isolated polynucleotide(s) set forth in (i); and (b) introducing at least one modification which is capable of modulating: (a) the expression of the NtLKR polynucleotide(s) in the Nicotiana tabacum plant; or (b) the activity of the NtLKR polypeptide(s) in the Nicotiana tabacum plant, as compared to a control in which the expression of the NtLKR polynucleotide(s) or the activity of the NtLKR polypeptide(s) has not been modified.

Suitably, in step (b) the at least one modification is introduced by genome editing; suitably, wherein the genome editing is selected from CRISPR-mediated genome editing, mutagenesis, zinc finger nuclease-mediated mutagenesis, chemical or radiation mutagenesis, homologous recombination, oligonucleotide-directed mutagenesis and meganuclease-mediated mutagenesis; or wherein in step (b) the at least one modification is introduced using an interference polynucleotide.

In a further aspect, there is disclosed a Nicotiana tabacum plant material obtained or obtainable by the method described above.

In a further aspect, there is disclosed a method of producing cured Nicotiana tabacum plant material having altered levels of at least one amino acid, comprising: (a) producing a Nicotiana tabacum plant as described above; (b) harvesting plant material (for example, leaf) from the Nicotiana tabacum plant; and (c) curing the plant material.

In a further aspect, there is disclosed cured Nicotiana tabacum plant material (for example, leaf) obtained or obtainable by the method described above.

In a further aspect, there is disclosed tobacco product comprising the Nicotiana tabacum plant material, the cured Nicotiana tabacum plant material, or the homogenised Nicotiana tabacum plant material described above or comprising the cured Nicotiana tabacum plant material described above.

Suitably, the tobacco product is a tobacco blend; suitably, wherein the tobacco blend comprises Virginia type tobacco and/or Burley type tobacco.

### Some Advantages

Advantageously, modulating the expression of a NtLKR polynucleotide(s) or the activity of a NtLKR polypeptide(s) can result in modulated levels of amino acids, especially in cured tobacco plant material. This can result in tobacco with novel flavour and/or sensory properties.

Advantageously, non-genetically modified plants can be created which may be more acceptable to consumers.

Advantageously, the present disclosure is not restricted to the use of ethyl methanesulfonate (EMS) mutant plants. An EMS mutant plant can have less potential to bring improved properties to a crop after breeding. Once breeding is started, the desirable characteristic(s) of the EMS mutant plant can be lost for different reasons. For example, several mutations may be required, the mutation can be dominant or recessive, and the identification of a point mutation in a gene target can be difficult to reach. In contrast, the present disclosure exploits the use of NtLKR that can be specifically manipulated to produce plants with a desirable phenotype.

Advantageously, no abnormal phenotype is observed, which renders the plants suitable for commercial production.

Down-regulation of asparagine synthetase (ASN) genes (WO2017042162; Bovet et al. (2019) Plants (Basel) 11; 8(11):492) also markedly alters the chemistry of tobacco without affecting the biomass. Advantageously, a combination of both ASN modulated plants and NtLKR modulated plants may rearrange the chemistry thereof—such as the amino acid chemistry of Burley or Dark or Virginia tobacco—and could further change the flavour and/or sensory properties thereof. Other genes and enzymes also play a role in the reorganization of amino acids and/or sugars during leaf yellowing—such as diaminopimelate aminotransferase (DAPAT), aspartate amino transferases (AAT) and chloroplast sulphate transporters (SULTR3) which may also change leaf chemistry. Modifying NtLKR expression or NtLKR activity together with the expression or activity of one or more of these other targets selected from one or more of ASN, DAPAT and AAT may be used to further modify the flavour and/or sensory properties of cured tobacco.

Advantageously, no significant differences in alkaloid content are seen which means that nicotine levels are not altered such that the same amount of nicotine can be delivered to the consumer of the tobacco.

### BRIEF DESCRIPTION OF THE DRAWINGS

FIG. 1 is two graphs which shows the identification of NtLKR-RNAi plants exhibiting low NtLKR expression via qRT-PCR. Relative expression of TN90 CT1-E438-4 n=4, CT1-E438-5 n=5, CT1-E438-6 n=5; T1-E438-2 n=4, T1-E438-5 n=5, T1-E438-11 n=5 are shown. Relative expression of Virgina K326 CT1-E437-5 n=5, CT1-E437-6 n=4, T1-E437-11 n=4, T1-E437-12 n=5, T1-E437-15 n=4 are shown. Tissues used for RNA isolation were green midrib/lamina.

FIG. 2 is a graph showing chlorophyll measures (CCI, 3 measures per plant) on C stalk position 21 days after nutritive solution switch.

FIG. 3 is three graphs which show the content of the three free amino acids Lys, Arg and Pro in the three independent NtLKR-RNAi lines (E438-2,-5-11) compared to WT (CT1-E438). Statistical analyses was performed with ANOVA, Tukey's HSD test.

FIG. 4 is six graphs showing the content of the three free amino acids Lys, Arg and Gln, the reducing sugars glucose and fructose and nitrate in the three independent NtLKR-RNAi lines (E43711,-12-15) compared to WT (CT1-E437). Statistical analyses was performed with ANOVA, Tukey's HSD test.

FIG. 5 is a graph showing the expression of NtLKR during early air-curing and early flue-curing.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. The materials, methods, and examples disclosed herein are illustrative only and not intended to be limiting.

The terms “comprise(s),” “include(s),” “having,” “has,” “can,” “contain(s),” and variants thereof are intended to be open-ended transitional phrases, terms, or words that do not preclude the possibility of additional acts or structures.

The singular forms “a,” “and” and “the” include plural references unless the context clearly dictates otherwise.

The present disclosure contemplates other embodiments “comprising,” “consisting of” and “consisting essentially of” the embodiments or elements presented herein, whether explicitly set forth or not.

For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used throughout the specification and the claims, the following terms have the following meanings: “Coding sequence” or “polynucleotide encoding” means the nucleotides (RNA or DNA molecule) that comprise a polynucleotide which encodes a polypeptide. The coding sequence can further include initiation and termination signals operably linked to regulatory elements including a promoter and polyadenylation signal capable of directing expression in the cells of an individual or mammal to which the polynucleotide is administered. The coding sequence may be codon optimized.

“Complement” or “complementary” can mean Watson-Crick (for example, A-T/U and C-G) or Hoogsteen base pairing between nucleotides or nucleotide analogues. “Complementarity” refers to a property shared between two polynucleotides, such that when they are aligned antiparallel to each other, the nucleotide bases at each position will be complementary.

“Construct” refers to a double-stranded, recombinant polynucleotide fragment comprising one or more polynucleotides. The construct comprises a “template strand” base-paired with a complementary “sense or coding strand.” A given construct can be inserted into a vector in two possible orientations, either in the same (or sense) orientation or in the reverse (or anti-sense) orientation with respect to the orientation of a promoter positioned within a vector—such as an expression vector.

The term “control” in the context of a control plant or control plant cells means a plant or plant cells in which the expression, function or activity of one or more genes or polypeptides has not been modified (for example, increased or decreased) and so it can provide a comparison with a plant in which the expression, function or activity of the same one or more genes or polypeptides has been modified. A “control plant” is a plant that is substantially equivalent to a test plant or modified plant in all parameters with the exception of the test parameters. For example, when referring to a plant into which a polynucleotide has been introduced, a control plant is an equivalent plant into which no such polynucleotide has been introduced. A control plant can be an equivalent plant into which a control polynucleotide has been introduced. In such instances, the control polynucleotide is one that is expected to result in little or no phenotypic effect on the plant. The control plant may comprise an empty vector. The control plant may correspond to a wild-type plant. The control plant may be a null segregant wherein the T1 segregant no longer possesses a transgene.

The term “decrease” or “decreased”, refers to a decrease of from about 10% to about 99%, or a decrease of at least 10%, at least 20%, at least 25%, at least 30%, at least 40%, at least 50%, at least 60%, at least 70%, at least 75%, at least 80%, at least 90%, at least 95%, at least 98%, at least 99%, or at least 100% or, or at least 150%, or at least 200% more of a quantity or a function—such as polypeptide function, transcriptional function, or polypeptide expression. The term “decreased,” or the phrase “a decreased amount” can refer to a quantity or a function that is less than what would be found in a plant or a product from the same variety of plant processed in the same manner, which has not been modified. Thus, in some contexts, a wild-type plant of the same variety that has been processed in the same manner is used as a control by which to measure whether a decrease in quantity is obtained.

“Donor DNA” or “donor template” refers to a double-stranded DNA fragment or molecule that includes at least a portion of the gene of interest. The donor DNA may encode a functional polypeptide.

“Endogenous gene or polypeptide” refers to a gene or polypeptide that originates from the genome of an organism and has not undergone a change, such as a loss, gain, or exchange of genetic material. An endogenous gene undergoes normal gene transmission and gene expression. An endogenous polypeptide undergoes normal expression.

“Enhancer sequences” refer to the sequences that can increase gene expression. These sequences can be located upstream, within introns or downstream of the transcribed region.

The transcribed region is comprised of the exons and the intervening introns, from the promoter to the transcription termination region. The enhancement of gene expression can be through various mechanisms including increasing transcriptional efficiency, stabilization of mature mRNA and translational enhancement.

“Expression” refers to the production of a functional product. For example, expression of a polynucleotide fragment may refer to transcription of the polynucleotide fragment (for example, transcription resulting in mRNA or functional RNA) or translation of mRNA into a precursor or mature polypeptide, or a combination thereof.

“Overexpression” refers to the production of a gene product in transgenic organisms that exceeds levels of production in a null segregating (or non-transgenic) organism from the same experiment.

“Functional” describes a polypeptide that has biological function or activity. A “functional gene” refers to a gene transcribed to mRNA, which is translated to a functional or active polypeptide.

“Genetic construct” refers to DNA or RNA molecules that comprise a polynucleotide that encodes a polypeptide. The coding sequence can include initiation and termination signals operably linked to regulatory elements including a promoter and polyadenylation signal capable of directing expression.

“Genome editing” generally refers to the process by which genomic nucleic acid in a cell is altered. This can be by removing, inserting or replacing one or more nucleotides in the genomic nucleic acid, for example. Endonucleases can be used to create specific breaks or nicks at defined locations in the genome and are further described herein.

The terms “homology” or “similarity” refer to the degree of sequence similarity between two polypeptides or between two polynucleotide molecules compared by sequence alignment.

The degree of homology between two discrete polynucleotides being compared is a function of the number of identical, or matching, nucleotides at comparable positions. Homology or similarity can be determined across the full length of a subject sequence.

“Identical” or “identity” in the context of two or more polynucleotides or polypeptides means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity.

In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be determined manually or by using a computer sequence algorithm such as ClustalW, ClustalX, BLAST, FASTA or Smith-Waterman. Suitable parameters for ClustalW maybe as follows: For polynucleotide alignments: Gap Open Penalty=15.0, Gap Extension Penalty=6.66, and Matrix=Identity. For polypeptide alignments: Gap Open Penalty=10. o, Gap Extension Penalty=0.2, and Matrix=Gonnet. For DNA and Protein alignments: ENDGAP=−1, and GAPDIST=4.

The term “increase” or “increased” refers to an increase of from about 10% to about 99%, or an increase of at least 10%, at least 20%, at least 25%, at least 30%, at least 40%, at least 50%, at least 60%, at least 70%, at least 75%, at least 80%, at least 90%, at least 95%, at least 98%, at least 99%, at least 100%, at least 150%, or at least 200% or more or more of a quantity or a function or an activity, such as but not limited to one or more of polypeptide function or activity, transcriptional function or activity and polypeptide expression. The term “increased,” or the phrase “an increased amount” can refer to a quantity or a function or an activity in a plant or a product generated from the plant that is more than what would be found in a plant or a product from the same variety of plant processed in the same manner, which has not been modified. Thus, in some contexts, a wild-type plant of the same variety that has been processed in the same manner is used as a control by which to measure whether an increase in quantity is obtained.

The term “inhibit” or “inhibited” refers to a decrease of from about 98% to about 100%, or a decrease of at least 98%, at least 99%, but particularly of 100%, of a quantity or a function or an activity, such as but not limited to one or more of polypeptide function or activity, transcriptional function or activity and polypeptide expression.

The term “introduced” can mean providing a polynucleotide (for example, a construct) or polypeptide into a cell. Introduced includes reference to the incorporation of a polynucleotide into a eukaryotic cell where the polynucleotide may be incorporated into the genome of the cell, and includes reference to the transient provision of a polynucleotide or polypeptide to the cell. Introduced includes reference to stable or transient transformation methods, as well as sexually crossing. Thus, “introduced” in the context of inserting a polynucleotide (for example, a recombinant construct/expression construct) into a cell, means “transfection” or “transformation” or “transduction” and includes reference to the incorporation of a polynucleotide into a eukaryotic cell where the polynucleotide may be incorporated into the genome of the cell (for example, chromosome, plasmid, plastid or mitochondrial DNA), converted into an autonomous replicon, or transiently expressed (for example, transfected mRNA).

The terms “isolated” or “purified” refer to material that is substantially or essentially free from components that normally accompany it as found in its native state. Purity and homogeneity are typically determined using analytical chemistry techniques such as polyacrylamide gel electrophoresis or high performance liquid chromatography. A polypeptide that is the predominant species present in a preparation is substantially purified. In particular, an isolated polynucleotide is separated from open reading frames that flank the desired gene and encode polypeptides other than the desired polypeptide. The term “purified” denotes that a polynucleotide or polypeptide gives rise to essentially one band in an electrophoretic gel.

Particularly, it means that the polynucleotide or polypeptide is at least 85% pure, more suitably at least 95% pure, and most suitably at least 99% pure. Isolated polynucleotides may be purified from a host cell in which they naturally occur. Conventional polynucleotide purification methods known to skilled artisans may be used to obtain isolated polynucleotides. The term also embraces recombinant polynucleotides and chemically synthesized polynucleotides.

“Liquid tobacco extract” describes the direct product of an extraction process carried out on a tobacco starting material. The extraction process for producing the liquid tobacco extract can comprise heating the tobacco starting material under specific heating conditions and collecting the volatile compounds generated. The liquid tobacco extract can contain a mixture of compounds that have derived from the tobacco starting material and have been removed during the extraction process, typically in combination with a liquid carrier or solvent.

“Modulate” or “modulating” refers to causing or facilitating a qualitative or quantitative change, alteration, or modification in a process, pathway, function or activity of interest. Without limitation, such a change, alteration, or modification may be an increase or decrease in the relative process, pathway, function or activity of interest. For example, gene expression or polypeptide expression or polypeptide function or activity can be modulated. Typically, the relative change, alteration, or modification will be determined by comparison to a control.

The term ‘non-naturally occurring’ describes an entity—such as a polynucleotide, a genetic mutation, a polypeptide, a plant, a plant cell and plant material—that is not formed by nature or that does not exist in nature. Such non-naturally occurring entities or artificial entities may be made, synthesized, initiated, modified, intervened, or manipulated by methods described herein or that are known in the art. Such non-naturally occurring entities or artificial entities may be made, synthesized, initiated, modified, intervened, or manipulated by man. Thus, a non-naturally occurring plant may not be produced using an essentially biological process. Thus, by way of example, a non-naturally occurring plant, a non-naturally occurring plant cell or non-naturally occurring plant material may be made using traditional plant breeding techniques—such as backcrossing—or by genetic manipulation technologies—such as antisense RNA, interfering RNA, meganuclease and the like. By way of further example, a non-naturally occurring plant, a non-naturally occurring plant cell or non-naturally occurring plant material may be made by introgression of or by transferring one or more genetic mutations (for example one or more polymorphisms) from a first plant or plant cell into a second plant or plant cell (which may itself be naturally occurring), such that the resulting plant, plant cell or plant material or the progeny thereof comprises a genetic constitution (for example, a genome, a chromosome or a segment thereof) that is not formed by nature or that does not exist in nature. The resulting plant, plant cell or plant material is thus artificial or non-naturally occurring. Accordingly, an artificial or non-naturally occurring plant or plant cell may be made by modifying a genetic sequence in a first naturally occurring plant or plant cell, even if the resulting genetic sequence occurs naturally in a second plant or plant cell that comprises a different genetic background from the first plant or plant cell. In certain embodiments, a mutation is not a naturally occurring mutation that exists naturally in a polynucleotide or a polypeptide—such as a gene or a polypeptide. Differences in genetic background can be detected by phenotypic differences or by molecular biology techniques known in the art—such as polynucleotide sequencing, presence or absence of genetic markers (for example, microsatellite RNA markers).

“Oligonucleotide” or “polynucleotide” means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a given sequence under stringent hybridization conditions. Thus, a polynucleotide also encompasses a probe that hybridizes under stringent hybridization conditions. Polynucleotides may be single stranded or double stranded, or may contain portions of both double stranded and single stranded sequence. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Polynucleotides may be obtained by chemical synthesis methods or by recombinant methods. The specificity of single-stranded DNA to hybridize complementary fragments is determined by the “stringency” of the reaction conditions (Sambrook et al., Molecular Cloning and Laboratory Manual, Second Ed., Cold Spring Harbor (1989)). To hybridize under “stringent conditions” describes hybridization protocols in which polynucleotides at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the given sequence hybridize to the given sequence at equilibrium. Since the given sequences are generally present at excess, at Tm, 50% of the probes are occupied at equilibrium.

Stringent conditions typically comprise: (1) low ionic strength and high temperature washes, for example 15 mM sodium chloride, 1.5 mM sodium citrate, 0.1% sodium dodecyl sulfate, at 50° C.; (2) a denaturing agent during hybridization, for example, 50% (v/v) formamide, 0.1% bovine serum albumin, 0.1% Ficoll, 0.1% polyvinylpyrrolidone, 50 mM sodium phosphate buffer (750 mM sodium chloride, 75 mM sodium citrate; pH 6.5), at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with a wash at 42° C. in 0.2×SSC (sodium chloride/sodium citrate) and 50% formamide at 55° C., followed by a high-stringency wash consisting of 0.1×SSC containing EDTA at 55° C. Suitably, the conditions are such that sequences at least about 65%, 70%, 75%, 85%, 90%, 95%, 98%, or 99% homologous to each other typically remain hybridized to each other.

“Moderately stringent conditions” use washing solutions and hybridization conditions that are less stringent, such that a polynucleotide will hybridize to the entire, fragments, derivatives, or analogs of the polynucleotide. One example comprises hybridization in 6×SSC, 5×Denhardt's solution, 0.5% SDS and 100 μg/mL denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (see Ausubel et al., Current Protocols in Molecular Biology, Volumes 1-3, John Wiley & Sons, Inc., Hoboken, N.J. (1993); Kriegler, Gene Transfer and Expression: A Laboratory Manual, Stockton Press, New York, N.Y. (1990); Perbal, A Practical Guide to Molecular Cloning, 2nd edition, John Wiley & Sons, New York, N.Y. (1988)). “Low stringent conditions” use washing solutions and hybridization conditions that are less stringent than those for moderate stringency, such that a polynucleotide will hybridize to the entire, fragments, derivatives, or analogs of the polynucleotide. A non-limiting example of low stringency hybridization conditions includes hybridization in 35% formamide, 5×SSC, 50 mM Tris HCl (pH 7.5), 5 mM EDTA, 0.02% PVP, 0.02% Ficoll, 0.2% BSA, 100 μg/mL denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross-species hybridizations, are well-described (see Ausubel et al., 1993; Kriegler, 1990).

“Operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. “Operably linked” refers to the association of polynucleotide fragments in a single fragment so that the function of one is regulated by the other. For example, a promoter is operably linked with a polynucleotide fragment when it is capable of regulating the transcription of that polynucleotide fragment.

The term “plant” refers to any plant at any stage of its life cycle or development, and its progenies. In one embodiment, the plant is a tobacco plant, which refers to a plant belonging to the genus Nicotiana. The term includes reference to whole plants, plant organs, plant tissues, plant propagules, plant seeds, plant cells and progeny of same. Plant cells include, without limitation, cells from seeds, suspension cultures, embryos, meristematic regions, callus tissue, leaves, roots, shoots, gametophytes, sporophytes, pollen, and microspores. Suitable species, cultivars, hybrids and varieties of tobacco plant are described herein.

“Plant material” includes leaf, root, sepal, root tip, petal, flower, shoot, stem, seed and stalk. Plant material can be viable or non-viable plant material.

“Polynucleotide”, “polynucleotide sequence” or “polynucleotide fragment” are used interchangeably herein and refer to a polymer of RNA or DNA that is single- or double-stranded, optionally containing synthetic, non-natural or altered nucleotide bases. The polynucleotides of the present disclosure are set forth in the accompanying sequence listing.

“Polypeptide” or “polypeptide sequence” refer to a polymer of amino acids in which one or more amino acid residues is an artificial chemical analogue of a corresponding naturally occurring amino acid, as well as to naturally occurring polymers of amino acids. The terms are also inclusive of modifications including, but not limited to, glycosylation, lipid attachment, sulfation, gamma-carboxylation of glutamic acid residues, hydroxylation and ADP-ribosylation. The polypeptides of the present disclosure are set forth in the accompanying sequence listing.

“Promoter” means a synthetic or naturally-derived molecule which is capable of conferring, activating or enhancing expression of a polynucleotide in a cell. The term refers to a polynucleotide element/sequence, typically positioned upstream and operably-linked to a double-stranded polynucleotide fragment. Promoters can be derived entirely from regions proximate to a native gene of interest, or can be composed of different elements derived from different native promoters or synthetic polynucleotide segments. A promoter may comprise one or more specific transcriptional regulatory sequences to further enhance expression, or to alter spatial expression or to alter temporal expression. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents.

“Tissue-specific promoter” and “tissue-preferred promoter” as used interchangeably herein refer to a promoter that is expressed predominantly but not necessarily exclusively in one tissue or organ, but that may also be expressed in one specific cell. A “developmentally regulated promoter” refers to a promoter whose function is determined by developmental events. A “constitutive promoter” refers to a promoter that causes a gene to be expressed in most cell types at most times. An “inducible promoter” selectively express an operably linked DNA sequence in response to the presence of an endogenous or exogenous stimulus, for example by chemical compounds (chemical inducers) or in response to environmental, hormonal, chemical or developmental signals or a combination of two or more thereof. Examples of inducible or regulated promoters include promoters regulated by light, heat, stress, flooding or drought, pathogens, phytohormones, wounding, or chemicals such as ethanol, jasmonate, salicylic acid, or safeners.

“Recombinant” refers to an artificial combination of two otherwise separated segments of sequence—such as by chemical synthesis or by the manipulation of isolated segments of polynucleotides by genetic engineering techniques. The term also includes reference to a cell or vector, that has been modified by the introduction of a heterologous polynucleotide or a cell derived from a cell so modified, but does not encompass the alteration of the cell or vector by naturally occurring events (for example, spontaneous mutation, natural transformation or transduction or transposition) such as those occurring without deliberate human intervention.

“Recombinant construct” refers to a combination of polynucleotides that are not normally found together in nature. Accordingly, a recombinant construct may comprise regulatory sequences and coding sequences that are derived from different sources, or regulatory sequences and coding sequences derived from the same source, but arranged in a manner different than that normally found in nature. The recombinant construct can be a recombinant DNA construct.

“Regulatory sequences” and “regulatory elements” as used interchangeably herein refer to polynucleotide sequences located upstream (5 non-coding sequences), within, or downstream (3′ non-coding sequences) of a coding sequence, and which influence the transcription, RNA processing or stability, or translation of the associated coding sequence. Regulatory sequences include promoters, translation leader sequences, introns, and polyadenylation recognition sequences. The terms “regulatory sequence” and “regulatory element” are used interchangeably herein.

The term “tobacco” is used in a collective sense to refer to tobacco crops (for example, a plurality of tobacco plants grown in the field and not hydroponically grown tobacco), tobacco plants and parts thereof, including but not limited to, roots, stems, leaves, flowers, and seeds prepared or obtained, as described herein. It is understood that “tobacco” refers to plants that belong to the Nicotiana genus and products thereof and includes Nicotiana tabacum plants and products thereof.

The term “tobacco products” refers to consumer tobacco products, including but not limited to, smoking materials (for example, cigarettes, cigars, and pipe tobacco), snuff, chewing tobacco, gum, and lozenges, as well as components, materials and ingredients for manufacture of consumer tobacco products. Suitably, these tobacco products are manufactured from tobacco leaves and stems harvested from tobacco and cut, dried, cured, or fermented according to conventional techniques in tobacco preparation.

“Transcription terminator”, “termination sequences”, or “terminator” refers to DNA sequences located downstream of a coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Transgenic” refers to any cell, cell line, callus, tissue, plant part or plant, the genome of which has been altered by the presence of a heterologous polynucleotide, such as a recombinant construct, including those initial transgenic events as well as those created by sexual crosses or asexual propagation from the initial transgenic event. The term does not encompass the alteration of the genome (chromosomal or extra-chromosomal) by conventional plant breeding methods or by naturally occurring events—such as random cross-fertilization, non-recombinant viral infection, non-recombinant bacterial transformation, non-recombinant transposition, or spontaneous mutation. Thus, in embodiments, the transgenic plant or part thereof is not produced using an essentially biological process.

“Transgenic plant” refers to a plant which comprises within its genome one or more heterologous polynucleotides, that is, a plant that contains recombinant genetic material not normally found therein and which has been introduced into the plant in question (or into progenitors of the plant) by human (artificial) manipulation. For example, the heterologous polynucleotide can be stably integrated within the genome such that the polynucleotide is passed on to successive generations. The heterologous polynucleotide can be integrated into the genome alone or as part of a recombinant construct. The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems and the like. Each heterologous polynucleotide may confer a different trait to the transgenic plant.

“Transgene” refers to a gene or genetic material containing a gene sequence that has been isolated from one organism and is introduced into a different organism. This non-native segment of DNA may retain the ability to produce RNA or polypeptide in the transgenic organism, or it may alter the normal function of the transgenic organism's genetic code.

“Variant” with respect to a polynucleotide means: (i) a portion or fragment of a polynucleotide; (ii) the complement of a polynucleotide or portion thereof; (iii) a polynucleotide that is substantially identical to a polynucleotide of interest or the complement thereof; or (iv) a polynucleotide that hybridizes under stringent conditions to the polynucleotide of interest, complement thereof, or a polynucleotide substantially identical thereto.

“Variant” with respect to a peptide or polypeptide means a peptide or polypeptide that differs in sequence by the insertion, deletion, or conservative substitution of amino acids, but retain at least one biological function or activity. Variant may also mean a polypeptide that retains at least one biological function or activity. A conservative substitution of an amino acid, that is, replacing an amino acid with a different amino acid of similar properties (for example, hydrophilicity, degree and distribution of charged regions) is recognized in the art as typically involving a minor change.

The term “variety” refers to a population of plants that share constant characteristics which separate them from other plants of the same species. While possessing one or more distinctive traits, a variety is further characterized by a very small overall variation between individuals within that variety. A variety is often sold commercially.

“Vector” refers to a polynucleotide vehicle that comprises a combination of polynucleotide components for enabling the transport of polynucleotides, polynucleotide constructs and polynucleotide conjugates and the like. A vector may be a viral vector, bacteriophage, bacterial artificial chromosome or yeast artificial chromosome. A vector may be a DNA or RNA vector. Suitable vectors include episomes capable of extra-chromosomal replication such as circular, double-stranded nucleotide plasmids; linearized double-stranded nucleotide plasmids; and other vectors of any origin. An “expression vector” is a polynucleotide vehicle that comprises a combination of polynucleotide components for enabling the expression of polynucleotide(s), polynucleotide constructs and polynucleotide conjugates and the like. Suitable expression vectors include episomes capable of extra-chromosomal replication such as circular, double-stranded nucleotide plasmids; linearized double-stranded nucleotide plasmids; and other functionally equivalent expression vectors of any origin. An expression vector comprises at least a promoter positioned upstream and operably-linked to a polynucleotide, polynucleotide constructs or polynucleotide conjugate, as defined below.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, plant biology, microbiology, genetics and polypeptide and polynucleotide chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

An isolated polynucleotide is disclosed comprising, consisting or consisting essentially of a sequence having at least 60% sequence identity to any of the sequences described herein, including any of polynucleotides shown in the sequence listing. Suitably, the isolated polynucleotide comprises, consists or consists essentially of a sequence having at least 60%, 61%, 62%, 63%, 64%, 65%, 66%, 67%, 68%, 69%, 70%, 75%, 80%, 85%, 86%, 87%, 88%, 89%, 90%, 91%, 92%, 93%, 94%, 95%, 96%, 97%, 98%, 99% or 100% sequence identity thereto. Suitably, the isolated polynucleotide comprises, consists or consists essentially of a sequence having at least 86%, 87%, 88%, 89%, 90%, 91%, 92%, 93%, 94%, 95%, 96%, 97%, 98%, 99% or 100% sequence identity thereto. Suitably, the isolated polynucleotide comprises, consists or consists essentially of a sequence having at least 88%, 89%, 90%, 91%, 92%, 93%, 94%, 95%, 96%, 97%, 98%, 99% or 100% sequence identity thereto.

Suitably, the polynucleotide(s) described herein encode an active polypeptide that has at least about 50%, 60%, 70%, 80%, 90%, 95%, 96%, 97%, 98%, 99%, 100% or more of the LKR function or activity of the polypeptide(s) shown in the sequence listing.

In another embodiment, there is provided an isolated LKR polynucleotide from Nicotiana tabacum (NtLKR) comprising, consisting or consisting essentially of a polynucleotide having at least 88%, 89%, 90%, 91%, 92%, 93%, 94%, 95%, 96%, 97%, 98%, 99% or 100% sequence identity to SEQ ID NO: 1 (NtLKR-S), or at least 86%, 87%, 88%, 89%, 90%, 91%, 92%, 93%, 94%, 95% 96%, 97%, 98%, 99% or 100% sequence identity to SEQ ID NO: 3 (NtLKR-T).

In another embodiment, there is provided a polynucleotide comprising, consisting or consisting essentially of a polynucleotide with substantial homology (that is, sequence similarity) or substantial identity to SEQ ID NO: 1 or SEQ ID NO: 3.

In another embodiment, there is provided fragments of SEQ ID NO: 1 or SEQ ID NO: 3 with substantial homology (that is, sequence similarity) or substantial identity thereto that have at least about 60%, 61%, 62%, 63%, 64%, 65%, 66%, 67%, 68%, 69%, 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO: 1 or SEQ ID NO: 3.

In another embodiment, there is provided fragments of SEQ ID NO: 1 with substantial homology (that is, sequence similarity) or substantial identity thereto that have at least about 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO: 1.

In another embodiment, there is provided fragments of SEQ ID NO: 3 with substantial homology (that is, sequence similarity) or substantial identity thereto that have at least about 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO: 3.

In another embodiment, there is provided polynucleotides comprising a sufficient or substantial degree of identity or similarity to SEQ ID NO: 1 or SEQ ID NO: 3 that encode a polypeptide that functions as an LKR.

In another embodiment, there is provided a polymer of polynucleotides which comprises, consists or consists essentially of a polynucleotide designated herein as SEQ ID NO: 1 or SEQ ID NO: 3.

Suitably, the polynucleotides described herein encode NtLKR polypeptides that have LKR activity.

A polynucleotide can include a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA or a fragment(s) thereof. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions or a fragment(s) thereof. In addition, the polynucleotide can be composed of triple-stranded regions comprising DNA, RNA, or both or a fragment(s) thereof. A polynucleotide can contain one or more modified bases, such as phosphothioates, and can be a peptide nucleic acid. Generally, polynucleotides can be assembled from isolated or cloned fragments of cDNA, genomic DNA, oligonucleotides, or individual nucleotides, or a combination of the foregoing. Although the polynucleotides described herein are shown as DNA sequences, they include their corresponding RNA sequences, and their complementary (for example, completely complementary) DNA or RNA sequences, including the reverse complements thereof.

Fragments of a polynucleotide may range from at least about 25 nucleotides, about 50 nucleotides, about 75 nucleotides, about 100 nucleotides about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1100 nucleotides, about 1200 nucleotides, about 1300 nucleotides or about 1400 nucleotides and up to the full-length polynucleotide encoding the polypeptides described herein.

A polynucleotide will generally contain phosphodiester bonds, although in some cases, polynucleotide analogues are included that may have alternate backbones, comprising, for example, phosphoramidate, phosphorothioate, phosphorodithioate, or O-methylphophoroamidite linkages; and peptide polynucleotide backbones and linkages. Other analogue polynucleotides include those with positive backbones; non-ionic backbones, and non-ribose backbones. Modifications of the ribose-phosphate backbone may be done for a variety of reasons, for example, to increase the stability and half-life of such molecules in physiological environments or as probes on a biochip. Mixtures of naturally occurring polynucleotides and analogues can be made; alternatively, mixtures of different polynucleotide analogues, and mixtures of naturally occurring polynucleotides and analogues may be made.

A variety of polynucleotide analogues are known, including, for example, phosphoramidate, phosphorothioate, phosphorodithioate, O-methylphophoroamidite linkages and peptide polynucleotide backbones and linkages. Other analogue polynucleotides include those with positive backbones, non-ionic backbones and non-ribose backbones. Polynucleotides containing one or more carbocyclic sugars are also included.

Other analogues include peptide polynucleotides which are peptide polynucleotide analogues. Among the uses of the disclosed polynucleotides, and fragments thereof, is the use of fragments as probes in hybridisation assays or primers for use in amplification assays. Such fragments generally comprise at least about 10, 11, 12, 13, 14, 15, 16, 17, 18, 19 or 20 or more contiguous nucleotides of a DNA sequence. In other embodiments, a DNA fragment comprises at least about 10, 15, 20, 30, 40, 50 or 60 or more contiguous nucleotides of a DNA sequence. Thus, in one aspect, there is also provided a method for detecting a polynucleotide comprising the use of the probes or primers or both.

The basic parameters affecting the choice of hybridization conditions and guidance for devising suitable conditions are described by Sambrook, J., E. F. Fritsch, and T. Maniatis (1989, Molecular Cloning: A Laboratory Manual, Cold Spring Harbor Laboratory Press, Cold Spring Harbor, N.Y.). Using knowledge of the genetic code in combination with the polypeptide sequences described herein, sets of degenerate oligonucleotides can be prepared. Such oligonucleotides are useful as primers, for example, in polymerase chain reactions (PCR), whereby DNA fragments are isolated and amplified.

At least one modification (for example, mutation) can be included in one or more of SEQ ID NO: 1 or SEQ ID NO: 3.

There is provided an isolated LKR polypeptide encoded by the polynucleotide(s) described herein.

There is provided an isolated LKR polypeptide comprising, consisting or consisting essentially of a polypeptide having at least 60% sequence identity to any of the polypeptides described herein, including any of the polypeptides shown in the sequence listing. Suitably, the isolated polypeptide comprises, consists or consists essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity thereto. Suitably, the isolated LKR polypeptide comprises, consists or consists essentially of a sequence having at least 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% sequence identity thereto. Suitably, the isolated LKR polypeptide comprises, consists or consists essentially of a sequence having at least 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% sequence identity thereto.

There is also provided a LKR polypeptide comprising, consisting or consisting essentially of a sequence having at least 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO: 2 (NtLKR-S).

There is also provided a LKR polypeptide comprising, consisting or consisting essentially of a sequence having at least 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO: 4 (NtLKR-T).

There is also provided a LKR polypeptide comprising, consisting or consisting essentially of a sequence having at least 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO: 2 or SEQ ID NO: 4. There is also provided a polypeptide encoded by SEQ ID NO: 2 or SEQ ID NO: 4.

The polypeptide can include sequences comprising a sufficient or substantial degree of identity or similarity to SEQ ID NO: 2 or SEQ ID NO: 4 to function as an LKR.

Fragment of the polypeptides described herein are also contemplated. The fragments of the polypeptide(s) typically retain some or all of the function or activity of the full length sequence—such as LKR activity. Fragments of a polypeptide may range from at least about 25 amino acids, about 50 amino acids, about 75 amino acids, about 100 amino acids about 150 amino acids, about 200 amino acids, about 250 amino acids, about 300 amino acids, about 400 amino acids, about 500 amino acids, and up to the full-length polypeptide described herein.

The polypeptides also include mutants produced by introducing any type of alterations (for example, insertions, deletions, or substitutions of amino acids; changes in glycosylation states; changes that affect refolding or isomerization, three-dimensional structures, or self-association states), which can be deliberately engineered or isolated naturally provided that they still have some or all of their function or activity. Suitably, this function or activity is modulated.

A deletion refers to removal of one or more amino acids from a polypeptide. An insertion refers to one or more amino acid residues being introduced into a predetermined site in a polypeptide. Insertions may comprise intra-sequence insertions of single or multiple amino acids. A substitution refers to the replacement of amino acids of the polypeptide with other amino acids having similar properties (such as similar hydrophobicity, hydrophilicity, antigenicity, propensity to form or break a-helical structures or β-sheet structures). Amino acid substitutions are typically of single residues, but may be clustered depending upon functional constraints placed upon the polypeptide and may range from about 1 to about 10 amino acids. The amino acid substitutions are suitably conservative amino acid substitutions as described below. Amino acid substitutions, deletions or insertions can be made using peptide synthetic techniques—such as solid phase peptide synthesis or by recombinant DNA manipulation. Methods for the manipulation of DNA sequences to produce substitution, insertion or deletion variants of a polypeptide are well known in the art. The variant may have alterations which produce a silent change and result in a functionally equivalent polypeptide. Deliberate amino acid substitutions may be made on the basis of similarity in polarity, charge, solubility, hydrophobicity, hydrophilicity and the amphipathic nature of the residues as long as the secondary binding of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example according to the Table below. Amino acids in the same block in the second column and suitably in the same line in the third column may be substituted for each other:

| | | |
|---|---|---|
| ALIPHATIC | Non-polar | Gly Ala Pro |
| | | Ile Leu Val |
| | Polar-uncharged | Cys Ser Thr Met |
| | | Asn Gly |
| | Polar-charged | Asp Glu |
| | | Lys Arg |
| AROMATIC | | His Phe TrpTyr |

The polypeptide may be a mature polypeptide or an immature polypeptide or a polypeptide derived from an immature polypeptide. Polypeptides may be in linear form or cyclized using known methods. Polypeptides typically comprise at least 10, at least 20, at least 30, or at least 40 contiguous amino acids.

At least one modification (for example, mutation) can be included in one or more of SEQ ID NO: 2 or SEQ ID NO: 4.

Recombinant constructs can be used to transform plants or plant cells in order to modulate polypeptide expression, function or activity. A recombinant polynucleotide construct can comprise a polynucleotide encoding one or more polynucleotides as described herein, operably linked to a regulatory region suitable for expressing the polypeptide. Thus, a polynucleotide can comprise a coding sequence that encodes the polypeptide as described herein. Plants or plant cells in which polypeptide expression, function or activity are modulated can include mutant, non-naturally occurring, transgenic, man-made or genetically engineered plants or plant cells. Suitably, the transgenic plant or plant cell comprises a genome that has been altered by the stable integration of recombinant DNA. Recombinant DNA includes DNA which has been genetically engineered and constructed outside of a cell and includes DNA containing naturally occurring DNA or cDNA or synthetic DNA. A transgenic plant can include a plant regenerated from an originally-transformed plant cell and progeny transgenic plants from later generations or crosses of a transformed plant. Suitably, the transgenic modification alters the expression or function or activity of the polynucleotide or the polypeptide described herein as compared to a control plant.

The polypeptide encoded by a recombinant polynucleotide can be a native polypeptide, or can be heterologous to the cell. In some cases, the recombinant construct contains a polynucleotide that modulates expression, operably linked to a regulatory region. Examples of suitable regulatory regions are described herein.

Vectors containing recombinant polynucleotide constructs such as those described herein are also provided. Suitable vector backbones include, for example, those routinely used in the art such as plasmids, viruses, artificial chromosomes, bacterial artificial chromosomes, yeast artificial chromosomes, or bacteriophage artificial chromosomes. Suitable expression vectors include, without limitation, plasmids and viral vectors derived from, for example, bacteriophage, baculoviruses, and retroviruses. Numerous vectors and expression systems are commercially available.

The vectors can include, for example, origins of replication, scaffold attachment regions or markers. A marker gene can confer a selectable phenotype on a plant cell. For example, a marker can confer biocide resistance, such as resistance to an antibiotic (for example, kanamycin, G418, bleomycin, or hygromycin), or an herbicide (for example, glyphosate, chlorsulfuron or phosphinothricin). In addition, an expression vector can include a tag sequence designed to facilitate manipulation or detection (for example, purification or localization) of the expressed polypeptide. Tag sequences, such as luciferase, beta-glucuronidase, green fluorescent polypeptide, glutathione S-transferase, polyhistidine, c-myc or hemagglutinin sequences typically are expressed as a fusion with the encoded polypeptide. Such tags can be inserted anywhere within the polypeptide, including at either the carboxyl or amino terminus.

A plant or plant cell can be transformed by having the recombinant polynucleotide integrated into its genome to become stably transformed. The plant or plant cell described herein can be stably transformed. Stably transformed cells typically retain the introduced polynucleotide with each cell division. A plant or plant cell can be transiently transformed such that the recombinant polynucleotide is not integrated into its genome. Transiently transformed cells typically lose all or some portion of the introduced recombinant polynucleotide with each cell division such that the introduced recombinant polynucleotide cannot be detected in daughter cells after a sufficient number of cell divisions.

A number of methods are available in the art for transforming a plant cell including biolistics, gene gun techniques, Agrobacterium-mediated transformation, viral vector-mediated transformation, freeze-thaw method, microparticle bombardment, direct DNA uptake, sonication, microinjection, plant virus-mediated transfer, and electroporation.

If a cell or cultured tissue is used as the recipient tissue for transformation, plants can be regenerated from transformed cultures if desired, by techniques known to those skilled in the art.

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a polynucleotide can be modulated in a similar manner. Some suitable regulatory regions initiate transcription only, or predominantly, in certain cell types. Methods for identifying and characterizing regulatory regions in plant genomic DNA are known in the art.

Exemplary promoters include tissue-specific promoters recognized by tissue-specific factors present in different tissues or cell types (for example, root-specific promoters, shoot-specific promoters, xylem-specific promoters), or present during different developmental stages, or present in response to different environmental conditions. Suitable promoters include constitutive promoters that can be activated in most cell types without requiring specific inducers. Examples of promoters that can be used to control polypeptide expression include the cauliflower mosaic virus 35S (CaMV/35S), SSU, OCS, lib4, usp, STLS1, B33, nos or ubiquitin- or phaseolin-promoters. Persons skilled in the art are capable of generating multiple variations of recombinant promoters.

Tissue-specific promoters are transcriptional control elements that are only active in particular cells or tissues at specific times during plant development, such as in vegetative tissues or reproductive tissues. Examples of tissue-specific promoters under developmental control include promoters that can initiate transcription only (or primarily only) in certain tissues, such as vegetative tissues, for example, roots or leaves, or reproductive tissues, such as fruit, ovules, seeds, pollen, pistols, flowers, or any embryonic tissue. Reproductive tissue-specific promoters may be, for example, anther-specific, ovule-specific, embryo-specific, endosperm-specific, integument-specific, seed and seed coat-specific, pollen-specific, petal-specific, sepal-specific, or combinations thereof.

Exemplary leaf-specific promoters include pyruvate, orthophosphate dikinase (PPDK) promoter from C4 plant (maize), cab-m1Ca+2 promoter from maize, the Arabidopsis thaliana myb-related gene promoter (Atmyb5), the ribulose biphosphate carboxylase (RBCS) promoters (for example, the tomato RBCS 1, RBCS2 and RBCS3A genes expressed in leaves and light-grown seedlings, RBCS1 and RBCS2 expressed in developing tomato fruits or ribulose bisphosphate carboxylase promoter expressed almost exclusively in mesophyll cells in leaf blades and leaf sheaths at high levels).

Exemplary senescence-specific promoters include a tomato promoter active during fruit ripening, senescence and abscission of leaves, a maize promoter of gene encoding a cysteine protease, the promoter of 82E4 and the promoter of SAG genes. Exemplary anther-specific promoters can be used. Exemplary root-preferred promoters known to persons skilled in the art may be selected. Exemplary seed-preferred promoters include both seed-specific promoters (those promoters active during seed development such as promoters of seed storage polypeptides) and seed-germinating promoters (those promoters active during seed germination).

Examples of inducible promoters include promoters responsive to pathogen attack, anaerobic conditions, elevated temperature, light, drought, cold temperature, or high salt concentration. Pathogen-inducible promoters include those from pathogenesis-related polypeptides (PR polypeptides), which are induced following infection by a pathogen (for example, PR polypeptides, SAR polypeptides, beta-1,3-glucanase, chitinase).

In addition to plant promoters, other suitable promoters may be derived from bacterial origin for example, the octopine synthase promoter, the nopaline synthase promoter and other promoters derived from Ti plasmids, or may be derived from viral promoters (for example, 35S and 19S RNA promoters of cauliflower mosaic virus (CaMV), constitutive promoters of tobacco mosaic virus, cauliflower mosaic virus (CaMV) 19S and 35S promoters, or figwort mosaic virus 35S promoter).

A plant or plant cell comprising at least one mutation in one or more polynucleotides or polypeptides as described herein is disclosed, wherein said mutation results in modulated function or activity of NtLKR or the polypeptide(s) encoded thereby.

There is provided a method for modulating the level of a NtLKR polypeptide in a (cured) plant or in (cured) plant material said method comprising introducing into the genome of said plant one or more mutations that modulate expression of at least one NtLKR, wherein said at least one NtLKR gene is selected from one or more of the NtLKR sequences according to the present disclosure.

There is also provided a method for identifying a plant with modulated levels of one or more amino acids in the plant or part thereof as compared to the level of the one or more amino acids in the control plant, said method comprising screening a polynucleotide sample from a plant of interest for the presence of one or more mutations in the NtLKR polynucleotide sequences according to the present disclosure, and optionally correlating the identified mutation(s) with mutation(s) that are known to modulate levels of one or more amino acids.

There is also disclosed a plant or plant cell that is heterozygous or homozygous for one or more mutations in a NtLKR gene according to the present disclosure, wherein said mutation results in modulated expression of the NtLKR gene or function or activity of the NtLKR polypeptide encoded thereby.

A number of approaches can be used to combine mutations in one plant including sexual crossing. A plant having one or more favourable heterozygous or homozygous mutations in a gene according to the present disclosure that modulates expression of the gene or the function or activity of the polypeptide encoded thereby can be crossed with a plant having one or more favourable heterozygous or homozygous mutations in one or more other genes that modulate expression thereof or the function or activity of the polypeptide encoded thereby. In one embodiment, crosses are made in order to introduce one or more favourable heterozygous or homozygous mutations within gene according to the present disclosure within the same plant. The function or activity of one or more polypeptides of the present disclosure in a plant is increased or decreased if the function or activity is lower or higher than the function or activity of the same polypeptide(s) in a plant that has not been modified to inhibit the function or activity of that polypeptide and which has been cultured, harvested and cured using the same protocols.

In some embodiments, the mutation(s) is introduced into a plant or plant cell using a mutagenesis approach, and the introduced mutation is identified or selected using methods known to those of skill in the art—such as Southern blot analysis, DNA sequencing, PCR analysis, or phenotypic analysis. Mutations that impact gene expression or that interfere with the function of the encoded polypeptide can be determined using methods that are well known in the art. Insertional mutations in gene exons usually result in null-mutants. Mutations in conserved residues can be particularly effective in inhibiting the metabolic function of the encoded polypeptide. It will be appreciated, for example, that a mutation in one or more of the highly conserved regions would likely alter polypeptide function, while a mutation outside of those highly conserved regions would likely have little to no effect on polypeptide function. In addition, a mutation in a single nucleotide can create a stop codon, which would result in a truncated polypeptide and, depending on the extent of truncation, loss of function.

Methods for obtaining mutant polynucleotides and polypeptides are also disclosed. Any plant of interest, including a plant cell or plant material can be genetically modified by various methods known to induce mutagenesis, including site-directed mutagenesis, oligonucleotide-directed mutagenesis, chemically-induced mutagenesis, irradiation-induced mutagenesis, mutagenesis utilizing modified bases, mutagenesis utilizing gapped duplex DNA, double-strand break mutagenesis, mutagenesis utilizing repair-deficient host strains, mutagenesis by total gene synthesis, DNA shuffling and other equivalent methods.

Mutations in the polynucleotides and polypeptides described herein can include man-made mutations or synthetic mutations or genetically engineered mutations. Mutations in the polynucleotides and polypeptides described herein can be mutations that are obtained or obtainable via a process which includes an in vitro or an in vivo manipulation step. Mutations in the polynucleotides and polypeptides described herein can be mutations that are obtained or obtainable via a process which includes intervention by man. The function or activity of the mutant polypeptide variant may be higher, lower or about the same as the unmutated polypeptide.

Methods that introduce a mutation randomly in a polynucleotide can include chemical mutagenesis and radiation mutagenesis. Chemical mutagenesis involves the use of exogenously added chemicals—such as mutagenic, teratogenic, or carcinogenic organic compounds—to induce mutations. Mutagens that create primarily point mutations and short deletions, insertions, missense mutations, simple sequence repeats, transversions ortransitions, including chemical mutagens or radiation, may be used to create the mutations.

Mutagens include ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Spontaneous mutations in the locus that may not have been directly caused by the mutagen are also contemplated provided that they result in the desired phenotype. Suitable mutagenic agents can also include, for example, ionising radiation—such as X-rays, gamma rays, fast neutron irradiation and UV radiation. The dosage of the mutagenic chemical or radiation is determined experimentally for each type of plant tissue such that a mutation frequency is obtained that is below a threshold level characterized by lethality or reproductive sterility. Any method of plant polynucleotide preparation known to those of skill in the art may be used to prepare the plant polynucleotide for mutation screening.

The mutation process may include one or more plant crossing steps.

After mutation, screening can be performed to identify mutations that create premature stop codons or otherwise non-functional genes. After mutation, screening can be performed to identify mutations that create functional genes that are capable of being expressed at increased or decreased levels. Screening of mutants can be carried out by sequencing, or by the use of one or more probes or primers specific to the gene or polypeptide. Specific mutations in polynucleotides can also be created that can result in modulated gene expression, modulated stability of mRNA, or modulated stability of polypeptide. Such plants are referred to herein as “non-naturally occurring” or “mutant” plants. Typically, the mutant or non-naturally occurring plants will include at least a portion of foreign or synthetic or man-made nucleotide (for example, DNA or RNA) that was not present in the plant before it was manipulated. The foreign nucleotide may be a single nucleotide, two or more nucleotides, two or more contiguous nucleotides or two or more non-contiguous nucleotides—such as at least 10, 20, 30, 40, 50, 100, 200, 300, 400, 500, 600, 700, 800, 900, 1000, 1100, 1200, 1300, 1400 or 1500 or more contiguous or non-contiguous nucleotides.

Sequence-specific polynucleotides that can interfere with the transcription of one or more endogenous gene(s); sequence-specific polynucleotides that can interfere with the translation of RNA transcripts (for example, double-stranded RNAs, siRNAs, ribozymes); sequence-specific polypeptides that can interfere with the stability of one or more polypeptides; sequence-specific polynucleotides that can interfere with the enzymatic function of one or more polypeptides or the binding function of one or more polypeptides with respect to substrates or regulatory polypeptides; antibodies that exhibit specificity for one or more polypeptides; small molecule compounds that can interfere with the stability of one or more polypeptides or the enzymatic function of one or more polypeptides or the binding function of one or more polypeptides; zinc finger polypeptides that bind one or more polynucleotides; and meganucleases that have function towards one or more polynucleotides can be used to modulate the expression or function or activity of one or more of the polynucleotides or polypeptides described herein. Genome editing technologies are well known in the art and are discussed further below.

Zinc finger polypeptides can be used to modulate the expression or function or activity of the one or more NtLKR polynucleotides described herein. The use of zinc finger nucleases is described in Nature Rev. Genet. (2010) 11 (9): 636-646).

Meganucleases, such as I-CreI, can be used to modulate the expression or function or activity of one or more of the NtLKR polynucleotides described herein. The use of meganucleases is described in Curr Gene Ther. (2011) February; 11(1):11-27 and Int J Mol Sci. (2019) 20(16), 4045.

Transcription activator-like effector nucleases (TALENs) can be used to modulate the expression or function or activity of one or more of the NtLKR polynucleotides described herein. The use of TALENs is described in Nature Rev. Mol. Cell Biol. (2013) 14: 49-55 and Int J Mol Sci. (2019) 20(16), 4045.

The CRISPR system can be used to modulate the expression or function or activity of one or more of the NtLKR polynucleotides described herein and is a preferred method. This technology is described in, for example, Plant Methods (2016) 12:8; Front Plant Sci. (2016) 7: 506; Biotechnology Advances (2015) 33, 1, p 41-52; Acta Pharmaceutica Sinica B (2017) 7, 3, p 292-302; Curr. Op. in Plant Biol. (2017) 36, 1-8 and Int J Mol Sci (2019) 20(16), 4045. As is well known in the art, the CRISPR editing system generally includes two components: a CRISPR-associated endonuclease (Cas) (for example, Cas9) and a guide RNA (gRNA). Cas forms a double stranded DNA break at a site in the genome that is defined by the sequence of a gRNA molecule bound to Cas. The location at which Cas breaks the DNA is defined by the unique sequence of the gRNA that is bound to it. gRNA is a specifically designed RNA sequence that recognizes the target DNA region of interest and directs the Cas nuclease there for editing. It has two sections: (i) a tracr RNA, which serves as a binding scaffold for the Cas nuclease; and (ii) crispr RNA (crRNA), a 17-20 nucleotide sequence complementary to the target DNA. The exact region of the DNA to be targeted will depend on the specific application. For example, to activate or repress a target polynucleotide, gRNAs can be targeted to the promoter driving expression of the target polynucleotide. Methods for designing gRNAs are well known in the art, including Chop Chop Harvard. The application of Cas9-based genome editing in Arabidopsis and tobacco is described in, for example, Methods Enzymol. (2014) 546:459-72 and Plant Physiol Biochem. (2018) 131:37-46. CRISPR technology has been widely implemented in plants (see, for example, WO2015/189693). In addition to Cas9, other RNA-guided nucleases for use in the CRISPR system have been described, including, CasI, CasIB, Cas2, Cas3, Cas4, Cas5, Cas6, Cas7, Cas8, CasIO, CpfI, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, CsxI7, CsxI4, CsxIO, Csx16, CsaX, Csx3, CsxI, CsxI5, CsfI, Csf2, Csf3 and Csf4. In certain embodiments, the use of Cas9 is preferred. The present disclosure further provides a CRISPR based genome editing system comprising an RNA-guided nuclease and a gRNA, where the CRISPR based genome editing system modulates the activity of one or more of the polynucleotides described herein. The present disclosure also provides a method of cleaving one or more polynucleotides in a plant cell, comprising introducing a gRNA and an RNA-guided nuclease into the plant cell, wherein the gRNA acts in association with the RNA-guided nuclease to create a strand break in one or more of the polynucleotides described herein. A CRISPR construct is also disclosed comprising: (i) a polynucleotide encoding a CRISPR-associated endonuclease; and (ii) a gRNA including a polynucleotide sequence (typically of about 17-20 nucleotides) complementary to the DNA of the polynucleotide as described herein that is to be targeted.

Antisense technology is another well-known method that can be used to modulate the expression or activity of one or more NtLKR polypeptides described herein. See, for example, Gene (1988) 10; 72(1-2):45-50.

NtLKR polynucleotides can be targeted for inactivation by introducing transposons (for example, IS elements or other mobile genetic elements) into the genomes of plants of interest. See, for example, Cytology and Genetics (2006) 40(4):68-81.

NtLKR polynucleotides can be targeted for inactivation by introducing ribozymes derived from a number of small circular RNAs that are capable of self-cleavage and replication in plants. See, for example, FEMS Microbiology Reviews (1999) 23, 3, 257-275.

The mutant or non-naturally occurring plants or plant cells can have any combination of one or more modifications (for example, mutations) in one or more of the NtLKR polynucleotides described herein which result in modulated expression or function or activity of those polynucleotides or their polynucleotide products. For example, the mutant or non-naturally occurring plants or plant cells may have a single modification in a single NtLKR polynucleotide or polypeptide; multiple modifications in a single NtLKR polynucleotide or polypeptide; a single modification in two or more NtLKR polynucleotides or polypeptides; or multiple modifications in two or more NtLKR polynucleotides or polypeptides. By way of further example, the mutant or non-naturally occurring plants or plant cells may have one or more modifications in a specific portion of NtLKR polynucleotide(s) or NtLKR polypeptide(s)—such as in a region of NtLKR that encodes an active site of the NtLKR polypeptide or a portion thereof. By way of further example, the mutant or non-naturally occurring plants or plant cells may have one or more modifications in a region outside of one or more NtLKR polynucleotide(s) or NtLKR polypeptide(s)—such as in a region upstream or downstream of the NtLKR polynucleotide(s) provided that it regulates the function or expression of the NtLKR. Upstream elements can include promoters, enhancers or transcription factors. Some elements—such as enhancers—can be positioned upstream or downstream of the gene it regulates. The element(s) need not be located near to the gene that it regulates since some elements have been found located several hundred thousand base pairs upstream or downstream of the gene that it regulates.

The mutant or non-naturally occurring plants or plant cells may have one or more modifications located within the first 100 nucleotides of the gene(s), within the first 200 nucleotides of the gene(s), within the first 300 nucleotides of the gene(s), within the first 400 nucleotides of the gene(s), within the first 500 nucleotides of the gene(s), within the first 600 nucleotides of the gene(s), within the first 700 nucleotides of the gene(s), within the first 800 nucleotides of the gene(s), within the first 900 nucleotides of the gene(s), within the first 1000 nucleotides of the gene(s), within the first 1100 nucleotides of the gene(s), within the first 1200 nucleotides of the gene(s), within the first 1300 nucleotides of the gene(s), within the first 1400 nucleotides of the gene(s) or within the first 1500 nucleotides of the gene(s). The mutant or non-naturally occurring plants or plant cells may have one or more modifications located within the first, second, third, fourth, fifth, sixth, seventh, eighth, ninth, tenth, eleventh, twelfth, thirteenth, fourteenth or fifteenth set of 100 nucleotides of the gene(s) or combinations thereof. Mutant or non-naturally occurring plants or plant cells (for example, mutant, non-naturally occurring or transgenic plants or plant cells and the like, as described herein) comprising the mutant polypeptide variants are disclosed.

In one embodiment, seeds from plants are mutagenised and then grown into first generation mutant plants. The first generation plants are then allowed to self-pollinate and seeds from the first generation plant are grown into second generation plants, which are then screened for mutations in their loci. Though the mutagenized plant material can be screened for mutations, an advantage of screening the second generation plants is that all somatic mutations correspond to germline mutations. One of skill in the art would understand that a variety of plant materials, including but not limited to, seeds, pollen, plant tissue or plant cells, may be mutagenised in order to create the mutant plants. However, the type of plant material mutagenised may affect when the plant polynucleotide is screened for mutations. For example, when pollen is subjected to mutagenesis prior to pollination of a non-mutagenized plant the seeds resulting from that pollination are grown into first generation plants. Every cell of the first generation plants will contain mutations created in the pollen; thus these first generation plants may then be screened for mutations instead of waiting until the second generation.

Prepared NtLKR polynucleotides from individual plants, plant cells, or plant material can optionally be pooled in order to expedite screening for mutations in the population of plants originating from the mutagenized plant tissue, cells or material. One or more subsequent generations of plants, plant cells or plant material can be screened. The size of the optionally pooled group is dependent upon the sensitivity of the screening method used. After the samples are optionally pooled, they can be subjected to polynucleotide-specific amplification techniques, such as PCR. Any one or more primers or probes specific to the gene or the sequences immediately adjacent to the gene may be utilized to amplify the sequences within the optionally pooled sample. Suitably, the one or more primers or probes are designed to amplify the regions of the locus where useful mutations are most likely to arise. Most suitably, the primer is designed to detect mutations within regions of the polynucleotide. Additionally, it is preferable for the primer(s) and probe(s) to avoid known polymorphic sites in order to ease screening for point mutations. To facilitate detection of amplification products, the one or more primers or probes may be labelled using any conventional labelling method. Primer(s) or probe(s) can be designed based upon the sequences described herein using methods that are well understood in the art. To facilitate detection of amplification products, the primer(s) or probe(s) may be labelled using any conventional labelling method. These can be designed based upon the sequences described herein using methods that are well understood in the art.

Polymorphisms may be identified by means known in the art and some have been described in the literature.

In some embodiments, a plant may be regenerated or grown from the plant, plant tissue or plant cell. Any suitable methods for regenerating or growing a plant from a plant cell or plant tissue may be used, such as, without limitation, tissue culture or regeneration from protoplasts. Suitably, plants may be regenerated by growing transformed plant cells on callus induction media, shoot induction media or root induction media. See, for example, McCormick et al., Plant Cell Reports 5:81-84 (1986). These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. Thus, “transformed seeds” refers to seeds that contain the nucleotide construct stably integrated into the plant genome.

Accordingly, in a further aspect there is provided a method of preparing a mutant plant. The method involves providing at least one cell of a plant comprising one or more NtLKR genes encoding a functional NtLKR. Next, the at least one cell of the plant is treated under conditions effective to modulate the function of the NtLKR polynucleotide(s). The at least one mutant plant cell is then propagated into a mutant plant, where the mutant plant has modulated levels of NtLKR polypeptide(s) described herein as compared to that of a control plant. In one embodiment of this method of making a mutant plant, the treating step involves subjecting the at least one cell to a chemical mutagenising agent as described above and under conditions effective to yield at least one mutant plant cell. In another embodiment of this method, the treating step involves subjecting the at least one cell to a radiation source under conditions effective to yield at least one mutant plant cell. The term “mutant plant” includes mutant plants in which the genotype is modified as compared to a control plant, suitably by means other than genetic engineering or genetic modification.

In certain embodiments, the mutant plant, mutant plant cell or mutant plant material may comprise one or more mutations that have occurred naturally in another plant, plant cell or plant material and confer a desired trait. This mutation can be incorporated (for example, introgressed) into another plant, plant cell or plant material (for example, a plant, plant cell or plant material with a different genetic background to the plant from which the mutation was derived) to confer the trait thereto. Thus by way of example, a mutation that occurred naturally in a first plant may be introduced into a second plant—such as a second plant with a different genetic background to the first plant. The skilled person is therefore able to search for and identify a plant carrying naturally in its genome one or more mutant alleles of the genes described herein which confer a desired trait. The mutant allele(s) that occurs naturally can be transferred to the second plant by various methods including breeding, backcrossing and introgression to produce a lines, varieties or hybrids that have one or more mutations in the genes described herein. The same technique can also be applied to the introgression of one or more non-naturally occurring mutation(s) from a first plant into a second plant. Plants showing a desired trait may be screened out of a pool of mutant plants. Suitably, the selection is carried out utilising the knowledge of the polynucleotide as described herein. Consequently, it is possible to screen for a genetic trait as compared to a control. Such a screening approach may involve the application of conventional amplification or hybridization techniques as discussed herein. Thus, a further aspect of the present disclosure relates to a method for identifying a mutant plant comprising: (a) providing a sample comprising one or more NtLKR polynucleotide(s) from a plant; and (b) determining the sequence of the polynucleotide(s), wherein a difference in the sequence of the polynucleotide(s) as compared to the polynucleotide(s) of a control plant is indicative that said plant is a mutant plant. In another aspect there is provided a method for identifying a mutant plant which accumulates increased or decreased levels of amino acids as compared to a control plant comprising: (a) providing a sample from a plant to be screened; (b) determining if said sample comprises one or more mutations in one or more NtLKR polynucleotides described herein; and (c) determining the level of at least one amino acid of said plant. Suitably the level of the at least one amino acid is determined in cured leaves. In another aspect there is provided a method for preparing a mutant plant which has increased or decreased levels of at least one amino acid—as compared to a control plant comprising: (a) providing a sample from a first plant; (b) determining if said sample comprises one or more mutations in one or more NtLKR polynucleotides described herein that result in modulated levels of the at least one amino acid; and (c) transferring the one or more mutations into a second plant. Suitably the level of the at least one amino acid is determined in cured leaves. The mutation(s) can be transferred into the second plant using various methods that are known in the art—such as by genetic engineering, genetic manipulation, introgression, plant breeding, backcrossing and the like. In one embodiment, the first plant is a naturally occurring plant. In one embodiment, the second plant has a different genetic background to the first plant. In another aspect there is provided a method for preparing a mutant plant which has increased or decreased levels of at least one amino acid as compared to a control plant comprising: (a) providing a sample from a first plant; (b) determining if said sample comprises one or more mutations in one or more of the NtLKR polynucleotides described herein that results in modulated levels of the at least one amino acid; and (c) introgressing the one or more mutations from the first plant into a second plant.

Suitably the level of the at least one amino acid is determined in cured leaves. In one embodiment, the step of introgressing comprises plant breeding, optionally including backcrossing and the like. In one embodiment, the first plant is a naturally occurring plant. In one embodiment, the second plant has a different genetic background to the first plant. In one embodiment, the first plant is not a cultivar or an elite cultivar. In one embodiment, the second plant is a cultivar or an elite cultivar. A further aspect relates to a mutant plant (including a cultivar or elite cultivar mutant plant) obtained or obtainable by the methods described herein. In certain embodiments, the mutant plant may have one or more mutations localised only to a specific region of the plant—such as within the sequence of the one or more NtLKR polynucleotide(s) described herein. According to this embodiment, the remaining genomic sequence of the mutant plant will be the same or substantially the same as the plant prior to the mutagenesis.

In certain embodiments, the mutant plants may have one or more mutations localised in more than one genomic region of the plant—such as within the sequence of one or more of the NtLKR polynucleotides described herein and in one or more further regions of the genome. According to this embodiment, the remaining genomic sequence of the mutant plant will not be the same or will not be substantially the same as the plant prior to the mutagenesis. In certain embodiments, the mutant plants may not have one or more mutations in one or more, two or more, three or more, four or more or five or more exons of the NtLKR polynucleotide(s) described herein; or may not have one or more mutations in one or more, two or more, three or more, four or more or five or more introns of the NtLKR polynucleotide(s) described herein; or may not have one or more mutations in a promoter of the NtLKR polynucleotide(s) described herein; or may not have one or more mutations in the 3′ untranslated region of the NtLKR polynucleotide(s) described herein; or may not have one or more mutations in the 5′ untranslated region of the NtLKR polynucleotide(s) described herein; or may not have one or more mutations in the coding region of the NtLKR polynucleotide(s) described herein; or may not have one or more mutations in the non-coding region of the NtLKR polynucleotide(s) described herein.

In a further aspect there is provided a method of identifying a plant, a plant cell or plant material comprising a mutation in a gene encoding a NtLKR polynucleotide described herein comprising: (a) subjecting a plant, a plant cell or plant material to mutagenesis; (b) obtaining a sample from said plant, plant cell or plant material or descendants thereof; and (c) determining the polynucleotide sequence of the NtLKR gene(s) or a variant or a fragment thereof, wherein a difference in said sequence is indicative of one or more mutations therein. This method also allows the selection of plants having mutation(s) that occur(s) in genomic regions that affect the expression of the NtLKR gene in a plant cell, such as a transcription initiation site, a start codon, a region of an intron, a boundary of an exon-intron, a terminator, Plants suitable for use in the present disclosure include monocotyledonous and dicotyledonous plants and plant cell systems and include members of the genera Nicotiana. Various embodiments are directed to mutant tobacco, non-naturally occurring tobacco or transgenic tobacco plants or tobacco plant cells and can be applied to any species of the genus Nicotiana, including N. rustica and N. tabacum (for example, LA B21, LN KY171, TI 1406, Basma, Galpao, Perique, Beinhart 1000-1, and Petico). Other species include N. acaulis, N. acuminata, N. africana, N. alata, N. ameghinoi, N. amplexicaulis, N. arentsii, N. attenuata, N. azambujae, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N. maritima, N. megalosiphon, N. miersii, N. noctiflora, N. nudicaulis, N. obtusifolia, N. occidentalis, N. occidentalis subsp. hesperis, N. otophora, N. paniculata, N. pauciflora, N. petunioides, N. plumbaginifolia, N. quadrivalvis, N. raimondii, N. repanda, N. rosulata, N. rosulata subsp. ingulba, N. rotundifolia, N. setchellii, N. simulans, N. solanifolia, N. spegazzinii, N. stocktonii, N. suaveolens, N. sylvestris, N. thyrsiflora, N. tomentosa, N. tomentosiformis, N. trigonophylla, N. umbratica, N. undulata, N. velutina, N. wigandioides, and N. x sanderae. In one embodiment, the plant is N. tabacum.

The use of tobacco cultivars and elite tobacco cultivars is also contemplated herein. The transgenic, non-naturally occurring or mutant plant may therefore be a tobacco variety or elite tobacco cultivar that comprises one or more transgenes, or one or more genetic mutations or a combination thereof. The genetic mutation(s) (for example, one or more polymorphisms) can be mutations that do not exist naturally in the individual tobacco variety or tobacco cultivar (for example, elite tobacco cultivar) or can be genetic mutation(s) that do occur naturally provided that the mutation does not occur naturally in the individual tobacco variety or tobacco cultivar (for example, elite tobacco cultivar).

Particularly useful Nicotiana tabacum varieties include Burley type, dark type, flue-cured type, and Oriental type tobaccos. Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF911, DT 538 LC Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KY14xL8 LC, Little Crittenden, McNair 373, McNair 944, msKY 14xL8, Narrow Leaf Madole, Narrow Leaf Madole LC, NBH 98, N-126, N-777LC, N-7371LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC, ‘Perique’ tobacco, PVHO3, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B13P, Xanthi (Mitchell-Mor), Bel-W3, 79-615, Samsun Holmes NN, KTRDC number 2 Hybrid 49, Burley 21, KY8959, KY9, MD 609, PG01, PGO4, P01, P02, P03, RG 11, RG 8, VA509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpão Comum, HB04P, Hicks Broadleaf, Kabakulak Elassona, Kutsage E1, LA BU 21, NC 2326, NC 297, PVH 2110, Red Russian, Samsun, Saplak, Simmaba, Talgar 28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

Embodiments are also directed to compositions and methods for producing mutant plants, non-naturally occurring plants, hybrid plants, or transgenic plants that have been modified to modulate the expression or function of one or more NtLKR polynucleotide(s) described herein (or any combination thereof as described herein). Advantageously, the mutant plants, non-naturally occurring plants, hybrid plants, or transgenic plants that are obtained may be similar or substantially the same in overall appearance to control plants. Various phenotypic characteristics such as degree of maturity, number of leaves per plant, stalk height, leaf insertion angle, leaf size (width and length), internode distance, and lamina-midrib ratio can be assessed by field observations.

One aspect relates to a seed of a mutant plant, a non-naturally occurring plant, a hybrid plant or a transgenic plant described herein. Suitably, the seed is a tobacco seed. A further aspect relates to pollen or an ovule of a mutant plant, a non-naturally occurring plant, a hybrid plant or a transgenic plant that is described herein. In addition, there is provided a mutant plant, a non-naturally occurring plant, a hybrid plant or a transgenic plant as described herein which further comprises a polynucleotide conferring male sterility.

Also provided is a tissue culture of regenerable cells of the mutant plant, non-naturally occurring plant, hybrid plant, or transgenic plant or a part thereof as described herein, which culture regenerates plants capable of expressing all the morphological and physiological characteristics of the parent. The regenerable cells include cells from leaves, pollen, embryos, cotyledons, hypocotyls, roots, root tips, anthers, flowers and a part thereof, ovules, shoots, stems, stalks, pith and capsules or callus or protoplasts derived therefrom.

The plant material that is described herein can be cured tobacco material. The CORESTA recommendation for tobacco curing is described in: CORESTA Guide N^{o} 17, April 2016, Sustainability in Leaf Tobacco Production.

The mutant, transgenic or non-naturally occurring plants or parts thereof of the present disclosure exhibit modulated levels of at least one amino acid in the plant material, for example, in cured leaves.

Suitably, the modulated levels of at least one amino acid are observed in at least cured leaves, suitably fully cured leaves. Tobacco is considered to be fully cured when the leaf's central rib is free of moisture, resulting in leaves that are light tan to reddish-brown to deep brown in colour.

Suitably, the cured leaves are taken from mid-position leaves of a plant. Suitably, there is no impact on the phenotype of the leaf with the modulated levels of at least one amino acid as compared to a leaf from a control plant.

In one embodiment, the level of lysine is increased as compared to a control plant or part thereof.

In one embodiment, the levels of lysine, arginine, glutamine, tyrosine, gamma aminobutyric acid (GABA) and alanine are increased as compared to a control plant or part thereof.

In one embodiment, the levels of lysine, arginine, glutamine, histidine, tyrosine, tryptophan, threonine, GABA, asparagine and alanine are increased as compared to a control plant or part thereof.

In one embodiment, the levels of lysine, arginine, proline, GABA, glutamine, leucine, alanine, phenylalanine, tyrosine and isoleucine are increased as compared to a control plant or part thereof.

In one embodiment, the levels of isoleucine, valine and serine are decreased as compared to a control plant or part thereof.

In one embodiment, the levels of methionine, threonine, and glycine are decreased as compared to a control plant or part thereof.

In one embodiment, the levels of aspartic acid and glutamic acid are not significantly altered as compared to a control plant or part thereof.

In one embodiment, the levels of proline, aspartic acid, leucine, phenylalanine, glutamic acid and methionine are not significantly altered as compared to a control plant or part thereof.

In one embodiment, the levels of asparagine, aspartic acid, tryptophan, histidine, glutamic acid serine and valine are not significantly altered as compared to a control plant or part thereof.

In one embodiment, the levels of total free amino acids are not significantly altered as compared to a control plant or part thereof.

In one embodiment, the plant is a Virgina tobacco plant in which the levels of lysine, arginine, glutamine, histidine, tyrosine, tryptophan, threonine, GABA, asparagine and alanine are increased as compared to a control plant or part thereof, suitably, wherein the increase is about a 4.6 fold increase for lysine, about a 2.81 fold increase for arginine, about a 2.06 fold increase for glutamine, about a 1.68 fold increase for histidine, about a 1.62 fold increase for tyrosine, about a 1.6 fold increase for tryptophan, about a 1.53 fold increase for threonine, about a 1.45 fold increase for GABA, about a 1.38 fold increase for asparagine and about a 1.3 fold increase for alanine as compared to a control plant or part thereof.

The Virgina tobacco plant can also have decreased levels of isoleucine, valine and serine as compared to a control plant or part thereof, suitably, wherein the decrease is about 39% for isoleucine, the decrease is about 17% for valine and the decrease is about 15% for serine as compared to the control plant or part thereof.

The Virgina tobacco plant can also has no significant change in the levels of proline, aspartic acid, leucine, phenylalanine, glutamic acid and methionine as compared to a control plant or part thereof.

The Virgina tobacco plant can have a decreased sum of sugars of about 15% (including a decrease in glucose, suitably a decrease of about 23%; a decrease in fructose, suitably a decrease of about 18%; and no significant difference in sucrose content) as compared to a control plant or part thereof.

The Virgina tobacco plant can have no significant difference in ammonia content as compared to a control plant or part thereof.

The Virgina tobacco plant can have a decreased nitrate content, suitably decreased by about 34%, as compared to a control plant or part thereof.

In another embodiment, the plant is a Burley tobacco plant in which the levels of lysine, arginine, proline, GABA, glutamine, leucine, alanine, phenylalanine, tyrosine and isoleucine are increased as compared to a control plant or part thereof, suitably, wherein the increase is about 11.2 fold for lysine, about 1.85 fold for arginine, about 1.41 fold proline, about 1.26 fold for GABA, about 1.25 fold for glutamine, about 1.24 fold for leucine, about 1.20 fold for alanine, about 1.15 fold for phenylalanine, about 1.09 fold for tyrosine and about 1.06 fold for isoleucine.

The Burley tobacco plant can have decreased levels of methionine, threonine and glycine as compared to a control plant or part thereof, suitably, wherein the decrease is about 29% for methionine, the decrease is about 10% for threonine and the decrease is about 8% for glycine as compared to the control plant or part thereof.

The Burley tobacco plant can have no significant change in the levels of asparagine, aspartic acid, tryptophan, histidine, glutamic acid, serine and valine as compared to a control plant or part thereof.

A further aspect relates to a mutant, non-naturally occurring or transgenic plant or cell in which the expression of one or more NtLKR polynucleotides or the activity of one or more NtLKR polypeptide(s) has been modulated, suitably decreased, that has increased levels of at least one amino acid (suitably, lysine) of at least a 4 fold or at least 11 fold as compared to a control plant or part thereof in which the expression of NtLKR or the activity of NtLKR has not been modulated, suitably decreased.

A still further aspect relates to cured plant material—such as cured leaf or cured tobacco—derived or derivable from the mutant, non-naturally occurring or transgenic plant or cell, wherein expression of one or more of the one or more NtLKR polynucleotides described herein or the function of the NtLKR polypeptide(s) encoded thereby is modulated, suitably decreased, and wherein the level of one or more amino acids (suitably, lysine) is modulated, suitably decreased, as compared to a control plant or part thereof.

Embodiments are also directed to compositions and methods for producing mutant, non-naturally occurring or transgenic plants or plant cells that have been modified to modulate, suitably decrease, the expression or activity of one or more of the NtLKR polynucleotides or NtLKR polypeptides described herein which can result in plants or plant parts (for example, leaves—such as cured leaves) or plant cells with modulated, suitably increased amino acid (suitably, lysine) content.

In one embodiment, the phenotype of the mutant, non-naturally occurring or transgenic plant is substantially the same as the control plant or part thereof. In one embodiment, the leaf weight of the mutant, non-naturally occurring or transgenic plant is substantially the same as the control plant or part thereof. In one embodiment, the leaf number of the mutant, non-naturally occurring or transgenic plant is substantially the same as the control plant or part thereof. In one embodiment, the leaf weight and the leaf number of the mutant, non-naturally occurring or transgenic plant is substantially the same as the control plant. In one embodiment, the stalk height of the mutant, non-naturally occurring or transgenic plants is substantially the same as the control plants or parts thereof at, for example, one, two or three or more months after field transplant or 10, 20, 30 or 36 or more days after topping. For example, the stalk height of the mutant, non-naturally occurring or transgenic plants is not less than the stalk height of the control plants or parts thereof. In another embodiment, the chlorophyll content of the mutant, non-naturally occurring or transgenic plants is decreased as compared to a control plant or part thereof. In another embodiment, the rate of leaf senescence in increased as compared to a control plant or part thereof.

In another aspect, there is provided a method for modulating the amount of at least one amino acid in at least a part of a plant (for example, the leaves—such as cured leaves) comprising: (i) modulating the expression or function of an one or more of the NtLKR polypeptides described herein, suitably, wherein the NtLKR polypeptide(s) is encoded by the corresponding NtLKR polynucleotides described herein; (ii) measuring the level of the at least one amino acid in at least a part (for example, the leaves—such as cured leaves—or tobacco or in smoke) of the mutant, non-naturally occurring or transgenic plant obtained in step (i); and (iii) identifying a mutant, non-naturally occurring or transgenic plant or part thereof in which the level of the at least one amino acid has been modulated in comparison to a control plant or part thereof.

In another aspect, there is provided a method for modulating the amount of at least one amino acid in cured plant material—such as cured leaf—comprising: (i) modulating the expression or function of an one or more of the NtLKR polypeptides (or any combination thereof as described herein), suitably, wherein the NtLKR polypeptide(s) is encoded by the corresponding NtLKR polynucleotides described herein; (ii) harvesting plant material—such as one or more of the leaves—and curing for a period of time; (iii) measuring the level of the at least one amino acid in cured plant material obtained in step (ii) or during step (ii); and (iv) identifying cured plant material in which the level of the at least one amino acid has been modulated in comparison to a control plant or part thereof.

An increase in expression as compared to the control may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200%, 300%, 500%, 1000% or more, which includes an increase in transcriptional function or NtLKR polynucleotide expression or NtLKR polypeptide expression.

An increase in function or activity as compared to a control may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200%, 300%, 500%, 1000% or more, which includes an increase in transcriptional function or NtLKR polynucleotide expression or NtLKR polypeptide expression or a combination thereof.

A decrease in expression as compared to a control may be from about 5% to about 100%, or a decrease of at least 10%, at least 20%, at least 25%, at least 30%, at least 40%, at least 50%, at least 60%, at least 70%, at least 75%, at least 80%, at least 90%, at least 95%, at least 98%, or 100%, which includes a decrease in transcriptional function or NtLKR polynucleotide expression or NtLKR polypeptide expression or a combination thereof.

A decrease in function or activity as compared to a control may be from about 5% to about 100%, or a decrease of at least 10%, at least 20%, at least 25%, at least 30%, at least 40%, at least 50%, at least 60%, at least 70%, at least 75%, at least 80%, at least 90%, at least 95%, at least 98%, or 100%, which includes a decrease in transcriptional function or NtLKR polynucleotide expression or NtLKR polypeptide expression or a combination thereof.

Polynucleotides and recombinant constructs described herein can be used to modulate the expression or function or activity of the NtLKR polynucleotides or NtLKR polypeptides described herein in a plant species of interest, suitably tobacco.

A number of polynucleotide based methods can be used to increase gene expression in plants and plant cells. By way of example, a construct, vector or expression vector that is compatible with the plant to be transformed can be prepared which comprises the gene of interest together with an upstream promoter that is capable of overexpressing the gene in the plant or plant cell. Exemplary promoters are described herein. Following transformation and when grown under suitable conditions, the promoter can drive expression in order to modulate the levels of NtLKR in the plant, or in a specific tissue thereof. In one exemplary embodiment, a vector carrying one or more NtLKR polynucleotides described herein (or any combination thereof as described herein) is generated to overexpress the gene in a plant or plant cell. The vector carries a suitable promoter—such as the cauliflower mosaic virus CaMV 35S promoter—upstream of the transgene driving its constitutive expression in all tissues of the plant. The vector also carries an antibiotic resistance gene in order to confer selection of the transformed calli and cell lines.

The expression of sequences from promoters can be enhanced by including expression control sequences, which are well known in the art. Signals associated with senescence and signals which are active during the curing procedure are specifically indicated.

Various embodiments are therefore directed to methods for modulating the expression level of one or more NtLKR polynucleotides described herein (or any combination thereof as described herein) by integrating multiple copies of the NtLKR polynucleotide(s) into a plant genome, comprising: transforming a plant cell host with an expression vector that comprises a promoter operably-linked to one or more NtLKR polynucleotides described herein. The polypeptide encoded by a recombinant polynucleotide can be a native polypeptide, or can be heterologous to the cell.

In one embodiment, the plant for use in the present disclosure is a mutant, non-naturally occurring or transgenic plant that is flue-cured.

In one embodiment, the plant for use in the present disclosure is a mutant, non-naturally occurring or transgenic plant that is sun-cured.

In one embodiment, the plant for use in the present disclosure is a mutant, non-naturally occurring or transgenic plant that is air-cured.

In one embodiment, the plant for use in the present disclosure is a mutant, non-naturally occurring or transgenic Virginia tobacco plant that is cured, for example, flue-cured.

In one embodiment, the plant for use in the present disclosure is a mutant, non-naturally occurring or transgenic Burley tobacco plant that is cured, for example, air-cured.

In one embodiment, the plant for use in the present disclosure is a mutant, non-naturally occurring or transgenic Dark tobacco plant that is cured, for example, fire-cured.

By modulating NtLKR expression and/or activity, the sensory profile of tobacco can be advantageously modified. For example, as can be appreciated from Table 5, RNAi modified Burely TN90 tobacco has a decreased harshness, makes the aerosol more round and smooth with less typical dark notes, but with more animalic and nutty notes (less trigeminals impact).

By way of further example, as can be appreciated from Table 6, RNAi modified Virginia K326 tobacco has increased hay notes with a rounder and darker flavour profile. A plant carrying a mutant allele of one or more NtLKR polynucleotides described herein can be used in a plant breeding program to create useful lines, varieties and hybrids. For example, a mutant allele can be introgressed into commercially important varieties described herein. Thus, methods for breeding plants are provided, that comprise crossing a mutant plant, a non-naturally occurring plant or a transgenic plant as described herein with a plant comprising a different genetic identity. The method may further comprise crossing the progeny plant with another plant, and optionally repeating the crossing until a progeny with the desirable genetic traits or genetic background is obtained. One purpose served by such breeding methods is to introduce a desirable genetic trait into other varieties, breeding lines, hybrids or cultivars, particularly those that are of commercial interest. Another purpose is to facilitate stacking of genetic modifications of different genes in a single plant variety, lines, hybrids or cultivars. Intraspecific as well as interspecific matings are contemplated. The progeny plants that arise from such crosses, also referred to as breeding lines, are examples of non-naturally occurring plants of the disclosure.

In one embodiment, a method is provided for producing a non-naturally occurring plant comprising: (a) crossing a mutant or transgenic plant with a second plant to yield progeny tobacco seed; (b) growing the progeny tobacco seed, under plant growth conditions, to yield the non-naturally occurring plant. The method may further comprises: (c) crossing the previous generation of non-naturally occurring plant with itself or another plant to yield progeny tobacco seed; (d) growing the progeny tobacco seed of step (c) under plant growth conditions, to yield additional non-naturally occurring plants; and (e) repeating the crossing and growing steps of (c) and (d) multiple times to generate further generations of non-naturally occurring plants.

The method may optionally comprises prior to step (a), a step of providing a parent plant which comprises a genetic identity that is characterized and that is not identical to the mutant or transgenic plant. In some embodiments, depending on the breeding program, the crossing and growing steps are repeated from 0 to 2 times, from 0 to 3 times, from 0 to 4 times, 0 to 5 times, from 0 to 6 times, from 0 to 7 times, from 0 to 8 times, from 0 to 9 times or from 0 to 10 times, in order to generate generations of non-naturally occurring plants. Backcrossing is an example of such a method wherein a progeny is crossed with one of its parents or another plant genetically similar to its parent, in order to obtain a progeny plant in the next generation that has a genetic identity which is closer to that of one of the parents. Techniques for plant breeding, particularly plant breeding, are well known and can be used in the methods of the disclosure. The disclosure further provides non-naturally occurring plants produced by these methods. Certain embodiments exclude the step of selecting a plant.

In some embodiments of the methods described herein, lines resulting from breeding and screening for variant genes are evaluated in the field using standard field procedures. Control genotypes including the original unmutagenized parent are included and entries are arranged in the field in a randomized complete block design or other appropriate field design. For tobacco, standard agronomic practices are used, for example, the tobacco is harvested, weighed, and sampled for chemical and other common testing before and during curing.

Statistical analyses of the data are performed to confirm the similarity of the selected lines to the parental line. Cytogenetic analyses of the selected plants are optionally performed to confirm the chromosome complement and chromosome pairing relationships.

DNA fingerprinting, single nucleotide polymorphism, microsatellite markers, or similar technologies may be used in a marker-assisted selection (MAS) breeding program to transfer or breed mutant alleles of a gene into other tobaccos, as described herein. For example, a breeder can create segregating populations from hybridizations of a genotype containing a mutant allele with an agronomically desirable genotype. Plants in the F2 or backcross generations can be screened using a marker developed from a genomic sequence or a fragment thereof, using one of the techniques listed herein. Plants identified as possessing the mutant allele can be backcrossed or self-pollinated to create a second population to be screened. Depending on the expected inheritance pattern or the MAS technology used, it may be necessary to self-pollinate the selected plants before each cycle of backcrossing to aid identification of the desired individual plants. Backcrossing or other breeding procedure can be repeated until the desired phenotype of the recurrent parent is recovered.

According to the disclosure, in a breeding program, successful crosses yield F1 plants that are fertile. Selected F1 plants can be crossed with one of the parents, and the first backcross generation plants are self-pollinated to produce a population that is again screened for variant gene expression (for example, the null version of the gene). The process of backcrossing, self-pollination, and screening is repeated, for example, at least 4 times until the final screening produces a plant that is fertile and reasonably similar to the recurrent parent. This plant, if desired, is self-pollinated and the progeny are subsequently screened again to confirm that the plant exhibits variant gene expression. In some embodiments, a plant population in the F2 generation is screened for variant gene expression, for example, a plant is identified that fails to express a polypeptide due to the absence of the gene according to standard methods, for example, by using a PCR method with primers based upon the polynucleotide sequence information for the polynucleotide(s) described herein (or any combination thereof as described herein).

Hybrid tobacco varieties can be produced by preventing self-pollination of female parent plants (that is, seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), or transgenic male sterility wherein a transgene inhibits microsporogenesis or pollen formation, or self-incompatibility. Female parent plants containing CMS are particularly useful. In embodiments in which the female parent plants are CMS, pollen is harvested from male fertile plants and applied manually to the stigmas of CMS female parent plants, and the resulting F1 seed is harvested.

Varieties and lines described herein can be used to form single-cross tobacco F1 hybrids. In such embodiments, the plants of the parent varieties can be grown as substantially homogeneous adjoining populations to facilitate natural cross-pollination from the male parent plants to the female parent plants. The F1 seed formed on the female parent plants is selectively harvested by conventional means. One also can grow the two parent plant varieties in bulk and harvest a blend of F1 hybrid seed formed on the female parent and seed formed upon the male parent as the result of self-pollination. Alternatively, three-way crosses can be carried out wherein a single-cross F1 hybrid is used as a female parent and is crossed with a different male parent. As another alternative, double-cross hybrids can be created wherein the F1 progeny of two different single-crosses are themselves crossed.

A population of mutant, non-naturally occurring or transgenic plants can be screened or selected for those members of the population that have a desired trait or phenotype. For example, a population of progeny of a single transformation event can be screened for those plants having a desired level of expression or function of the polypeptide(s) encoded thereby.

Physical and biochemical methods can be used to identify expression or activity levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme function of polypeptides and polynucleotides; and polypeptide gel electrophoresis, Western blots, immunoprecipitation, and enzyme-linked immunoassays to detect polypeptides. Other techniques such as in situ hybridization, enzyme staining, and immunostaining and enzyme assays also can be used to detect the presence or expression, function or activity of NtLKR polypeptides or polynucleotides.

Mutant, non-naturally occurring or transgenic plant cells and plants are described herein comprising one or more recombinant polynucleotides, one or more polynucleotide constructs, one or more double-stranded RNAs, one or more conjugates or one or more vectors/expression vectors.

Without limitation, the plants and parts thereof described herein can be modified either before or after the expression, function or activity of the one or more NtLKR polynucleotides or NtLKR polypeptides according to the present disclosure have been modulated.

One or more of the following further genetic modifications can be present in the mutant, non-naturally occurring or transgenic plants and parts thereof. One or more genes that are involved in the conversion of nitrogenous metabolic intermediates can be modified resulting in lower levels of at least one tobacco-specific nitrosamine (TSNA). Non-limiting examples of such genes include those encoding nicotine demethylase—such as CYP82E4, CYP82E5 and CYP82E10 as described in WO2006/091194, WO2008/070274, WO2009/064771 and WO2011/088180—and nitrate reductase, as described in WO2016/046288. One or more genes that are involved in heavy metal uptake or heavy metal transport can be modified resulting in lower heavy metal content. Non-limiting examples include genes in the family of multidrug resistance associated polypeptides, the family of cation diffusion facilitators (CDF), the family of Zrt-Irt-like polypeptides (ZIP), the family of cation exchangers (CAX), the family of copper transporters (COPT), the family of heavy-metal ATPases (for example, HMAs, as described in WO2009/074325 and WO2017/129739), the family of homologs of natural resistance-associated macrophage polypeptides (NRAMP), and other members of the family of ATP-binding cassette (ABC) transporters (for example, MRPs), as described in WO2012/028309, which participate in transport of heavy metals—such as cadmium.

Other exemplary modifications can result in plants with modulated expression or function of isopropylmalate synthase which results in a change in sucrose ester composition which can be used to alter favour profile (see WO2013/029799). Other exemplary modifications can result in plants with modulated expression or function of threonine synthase in which levels of methional can be modulated (see WO2013/029800). Other exemplary modifications can result in plants with modulated expression or function of one or more of neoxanthin synthase, lycopene beta cyclase and 9-cis-epoxycarotenoid dioxygenase to modulate beta-damascenone content to alter flavour profile (see WO2013/064499). Other exemplary modifications can result in plants with modulated expression or function of members of the CLC family of chloride channels to modulate nitrate levels therein (see WO2014/096283 and WO2015/197727). Other exemplary modifications can result in plants with modulated expression or function of one or more asparagine synthetases to modulate levels of asparagine in leaf and modulated levels of acrylamide in aerosol produced upon heating or combusting the leaf (see WO2017/129739). Other exemplary modifications can result in plants with modulated protease activity during curing (see WO2016/009006). Other exemplary modifications can result in plants having decreased nitrate levels by altering the gene expression of nitrate reductase (for example, Nia2) or the activity of the protein encoded thereby (see WO2016/046288). Other exemplary modifications can result in plants having modified alkaloid levels by altering the gene expression of putative ABC-2 transporters NtABCGI-T and NtABCGI-S or the activity of the protein encoded thereby (see WO2019/086609). Other exemplary modifications can result in plants having modulated time to flowering by altering the gene expression of genes encoding Terminal Flower 1 (TFL1) or the activity of the protein encoded thereby (see WO2018/114641). Other exemplary modifications can result in plants with modulated expression or function of one or more asparagine synthetases to modulate levels of asparagine in leaf and modulated levels of acrylamide in aerosol produced upon heating or combusting the leaf (see WO2017/042162). Examples of other modifications include modulating herbicide tolerance, for example, glyphosate is an active ingredient of many broad spectrum herbicides. Glyphosate resistant transgenic plants have been developed by transferring the aroA gene (a glyphosate EPSP synthetase from Salmonella typhimurium and E. coli). Sulphonylurea resistant plants have been produced by transforming the mutant ALS (acetolactate synthetase) gene from Arabidopsis. OB polypeptide of photosystem II from mutant Amaranthus hybridus has been transferred in to plants to produce atrazine resistant transgenic plants; and bromoxynil resistant transgenic plants have been produced by incorporating the bxn gene from the bacterium Klebsiella pneumoniae. Another exemplary modification results in plants that are resistant to insects. Bacillus thuringiensis (Bt) toxins can provide an effective way of delaying the emergence of Bt-resistant pests, as recently illustrated in broccoli where pyramided crylAc and cry1C Bt genes controlled diamondback moths resistant to either single polypeptide and significantly delayed the evolution of resistant insects. Another exemplary modification results in plants that are resistant to diseases caused by pathogens (for example, viruses, bacteria, fungi). Plants expressing the Xa21 gene (resistance to bacterial blight) with plants expressing both a Bt fusion gene and a chitinase gene (resistance to yellow stem borer and tolerance to sheath) have been engineered. Another exemplary modification results in altered reproductive capability, such as male sterility. Another exemplary modification results in plants that are tolerant to abiotic stress (for example, drought, temperature, salinity), and tolerant transgenic plants have been produced by transferring acyl glycerol phosphate enzyme from Arabidopsis; genes coding mannitol dehydrogenase and sorbitol dehydrogenase which are involved in synthesis of mannitol and sorbitol improve drought resistance. Another exemplary modification results in plants in which the activity of one or more nicotine N-demethylases is modulated such that the levels of nornicotine and metabolites of nornicotine—that are formed during curing can be modulated (see WO2015169927). Other exemplary modifications can result in plants with improved storage polypeptides and oils, plants with enhanced photosynthetic efficiency, plants with prolonged shelf life, plants with enhanced carbohydrate content, and plants resistant to fungi. Transgenic plants in which the expression of S-adenosyl-L-methionine (SAM) or cystathionine gamma-synthase (CGS), or a combination thereof, has been modulated are also contemplated. One or more genes that are involved in the nicotine synthesis pathway can be modified resulting in plants or parts of plants that when cured, produce modulated levels of nicotine. The nicotine synthesis genes can be selected from the group consisting of: A622, BBLa, BBLb, JRE5L1, JRE5L2, MATE1, MATE 2, MPO1, MPO2, MYC2a, MYC2b, NBB1, nic1, nic2, NUP1, NUP2, PMT1, PMT2, PMT3, PMT4 and OPT or a combination of one or more thereof. One or more genes that are involved in controlling the amount of one or more alkaloids can be modified resulting in plants or parts of plants that produce modulated levels of alkaloid. Alkaloid level controlling genes can be selected from the group consisting of; BBLa, BBLb, JRE5L1, JRE5L2, MATE1, MATE 2, MYC2a, MYC2b, nic1, nic2, NUP1 and NUP2 or a combination of two or more thereof.

Other exemplary modifications can result in plants with modulated amino acid content (see WO2019/185703 and WO2021/063863) or with modulated sugar content (see WO2019/185699 and WO2021/063860 and WO2021/063863) or with modulated nitrate levels (see WO2020/141062) or with modulated sugar and amino acid content (see WO2021/063863).

In a preferred embodiment, the further genetic modification concerns asparagine synthetase (ASN) genes as described in WO2017042162. Modulating the expression of ASN genes (for example, one or more of NtASN1-S, NtASN1-T, NtASN5-S and NtASN5-Tas described in WO2017042162) or the activity of ASN (for example, NtASN1-S, NtASN1-T, NtASN5-S and NtASN5-T as described in WO2017042162) markedly alters the chemistry of tobacco cured leaf without affecting the biomass. Accordingly, modulating the expression and/or activity of the combination of ASN and NtLKR may have the potential to rearrange the chemistry of cured tobacco leaf (particularly the amino acid chemistry of Burley or Dark tobacco) and thereby alter the sensory properties.

In addition to ASN, other genes and enzymes play a role in the reorganization of amino acids and/or sugars during leaf yellowing—such as diaminopimelate aminotransferase (DAPAT), which is involved in both catabolism and anabolism of lysine, and aspartate amino transferases (AAT), and which is expressed during senescence and has the potential to change leaf chemistry after curing (WO2019/185703). The chloroplast sulphate transporter SULTR3—such as NtSULTR3;1A-S, NtSULTR3;1A-T and NtSULTR3;3-T—play a role in sugar and amino acid metabolism during curing (see WO2021/063863). Accordingly, the further genetic modification can concern DAPAT and/or AAT (for example, one or more of NtAATI-S, NtAAT1-T, NtAA T2-S, NtAAT2-T, NtAA T3-S, NtAAT3-T, NtAA T4-S or NtAAT4-T as described in WO2017042162) and/or one or more of NtSULTR3;1A-S, NtSULTR3;1A-T and NtSULTR3;3-T as described in see WO2021/063863. Modulating the expression and/or activity of the combination of DAPAT and/or AAT and/or SULTR3 and NtLKR may have the potential to rearrange the chemistry of cured tobacco leaf and thereby alter the sensory properties. Modifications to combinations of NtLKR and one or more, or two or more, or three of more or four or more of ASN and DAPAT and AAT and SULTR3 are disclosed, including NtLKR and ASN; NtLKR and DAPAT; NtLKR and AAT; NtLKR and ASN and DAPAT; NtLKR and ASN and AAT; NtLKR and ASN and DAPAT and AAT; NtLKR and SULTR3; NtLKR and ASN and SULTR3; NtLKR and DAPAT and SULTR3; NtLKR and AAT and SULTR3; NtLKR and ASN and DAPAT and SULTR3; NtLKR and ASN and AAT and SULTR3; NtLKR and ASN and DAPAT and AAT and SULTR3.

One or more traits may be introgressed into the mutant, non-naturally occurring or transgenic plants from another cultivar or may be directly transformed into it.

Various embodiments provide mutant plants, non-naturally occurring plants or transgenic plants, as well as biomass in which the expression level of one or more polynucleotides according to the present disclosure are modulated to thereby modulate the level of polypeptide(s) encoded thereby.

Parts of the plants described herein, particularly the leaf lamina and/or stalks and/or midrib of such plants, can be incorporated into or used in making various consumable products including but not limited to aerosol forming materials, aerosol forming devices, smoking articles, smokable articles, smokeless products, medicinal or cosmetic products, intravenous preparations, tablets, powders, and tobacco products. Examples of aerosol forming materials include tobacco compositions, tobaccos, tobacco extract, cut tobacco, cut filler, cured tobacco, expanded tobacco, homogenized tobacco, reconstituted tobacco, and pipe tobaccos.

Smoking articles and smokable articles are types of aerosol forming devices. Examples of smoking articles or smokable articles include cigarettes, cigarillos, and cigars. Examples of smokeless products comprise chewing tobaccos, and snuffs. In certain aerosol forming devices, rather than combustion, a tobacco composition or another aerosol forming material is heated by one or more electrical heating elements to produce an aerosol. In another type of heated aerosol forming device, an aerosol is produced by the transfer of heat from a combustible fuel element or heat source to a physically separate aerosol forming material, which may be located within, around or downstream of the heat source. Smokeless tobacco products and various tobacco-containing aerosol forming materials may contain tobacco in any form, including as dried particles, shreds, granules, powders, or a slurry, deposited on, mixed in, surrounded by, or otherwise combined with other ingredients in any format, such as flakes, films, tabs, foams, or beads. The term ‘smoke’ is used to describe a type of aerosol that is produced by smoking articles, such as cigarettes, or by combusting an aerosol forming material.

In one embodiment, there is also provided cured plant material from the mutant, transgenic and non-naturally occurring plants described herein. Processes of curing green tobacco leaves are known by those having skills in the art and include without limitation air-curing, fire-curing, flue-curing and sun-curing as described herein.

In another embodiment, there is described tobacco products including tobacco-containing aerosol forming materials comprising plant material—such as leaves, suitably cured leaves—from the mutant tobacco plants, transgenic tobacco plants or non-naturally occurring tobacco plants described herein. The tobacco products described herein can be a blended tobacco product which may further comprise unmodified tobacco.

The mutant, non-naturally occurring or transgenic plants may have other uses in, for example, agriculture.

The disclosure also provides methods for producing seeds comprising cultivating the mutant plant, non-naturally occurring plant, or transgenic plant described herein, and collecting seeds from the cultivated plants. Seeds from plants described herein can be conditioned and bagged in packaging material by means known in the art to form an article of manufacture. Packaging material such as paper and cloth are well known in the art. A package of seed can have a label, for example, a tag or label secured to the packaging material, a label printed on the package that describes the nature of the seeds therein.

Compositions, methods and kits for genotyping plants for identification, selection, or breeding can comprise a means of detecting the presence of a NtLKR polynucleotide(s) in a sample of polynucleotide. Accordingly, a composition is described comprising one or more primers for specifically amplifying at least a portion of one or more of the NtLKR polynucleotides and optionally one or more probes and optionally one or more reagents for conducting the amplification or detection.

Accordingly, gene specific oligonucleotide primers or probes comprising about 10 or more contiguous polynucleotides corresponding to the NtLKR polynucleotide(s) described herein are disclosed. Said primers or probes may comprise or consist of about 15, 20, 25, 30, 40, 45 or 50 more contiguous polynucleotides that hybridise (for example, specifically hybridise) to the NtLKR polynucleotide(s) described herein. In some embodiments, the primers or probes may comprise or consist of about 10 to 50 contiguous nucleotides, about 10 to 40 contiguous nucleotides, about 10 to 30 contiguous nucleotides or about 15 to 30 contiguous nucleotides that may be used in sequence-dependent methods of gene identification (for example, Southern hybridization) or isolation (for example, in situ hybridization of bacterial colonies or bacteriophage plaques) or gene detection (for example, as one or more amplification primers in amplification or detection). The one or more specific primers or probes can be designed and used to amplify or detect a part or all of the polynucleotide(s). By way of specific example, two primers may be used in a PCR protocol to amplify a polynucleotide fragment. The PCR may also be performed using one primer that is derived from a polynucleotide sequence and a second primer that hybridises to the sequence upstream or downstream of the polynucleotide sequence—such as a promoter sequence, the 3′ end of the mRNA precursor or a sequence derived from a vector. Examples of thermal and isothermal techniques useful for in vitro amplification of polynucleotides are well known in the art. The sample may be or may be derived from a plant, a plant cell or plant material or a tobacco product made or derived from the plant, the plant cell or the plant material as described herein.

In a further aspect, there is also provided a method of detecting a NtLKR polynucleotide(s) described herein (or any combination thereof as described herein) in a sample comprising the step of: (a) providing a sample comprising, or suspected of comprising, a polynucleotide; (b) contacting said sample with one or more primers or one or more probes for specifically detecting at least a portion of the NtLKR polynucleotide(s); and (c) detecting the presence of an amplification product, wherein the presence of an amplification product is indicative of the presence of the NtLKR polynucleotide(s) in the sample. In a further aspect, there is also provided the use of one or more primers or probes for specifically detecting at least a portion of the NtLKR polynucleotide(s). Kits for detecting at least a portion of the NtLKR polynucleotide(s) are also provided which comprise one or more primers or probes for specifically detecting at least a portion of the NtLKR polynucleotide(s). The kit may comprise reagents for polynucleotide amplification—such as PCR—or reagents for probe hybridization-detection technology—such as Southern Blots, Northern Blots, in-situ hybridization, or microarray. The kit may comprise reagents for antibody binding-detection technology such as Western Blots, ELISAs, SELDI mass spectrometry or test strips. The kit may comprise reagents for DNA sequencing. The kit may comprise reagents and instructions for using the kit.

In some embodiments, a kit may comprise instructions for one or more of the methods described. The kits described may be useful for genetic identity determination, phylogenetic studies, genotyping, haplotyping, pedigree analysis or plant breeding particularly with co-dominant scoring.

The present disclosure also provides a method of genotyping a plant, a plant cell or plant material comprising a NtLKR polynucleotide as described herein. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. The specific method of genotyping may employ any number of molecular marker analytic techniques including amplification fragment length polymorphisms (AFLPs). AFLPs are the product of allelic differences between amplification fragments caused by polynucleotide variability. Thus, the present disclosure further provides a means to follow segregation of one or more genes or polynucleotides as well as chromosomal sequences genetically linked to these genes or polynucleotides using such techniques as AFLP analysis.

There is also disclosed herein methods of producing a liquid tobacco extract and a liquid tobacco extract produced by the method(s).

A specific extraction temperature is selected for the tobacco starting material. The extraction temperature(s) is typically selected from within the range of about 100 degrees Celsius to about 160 degrees Celsius. The duration of the heating step may optionally be controlled to provide a degree of control over the composition of the extract derived from the tobacco starting material(s). Suitably, the tobacco starting material(s) is heated at the extraction temperature for at least about 90 minutes, more suitably at least about 120 minutes. The heating step is typically carried out in an inert atmosphere. Suitably, a flow of an inert gas—such as nitrogen—is passed through the starting tobacco material during the heating step. The volatile tobacco compounds are released into the flow of inert gas during the heating step such that the inert gas acts as a carrier for the volatile components. The flow of inert gas can be at a flow rate of at least about 25 litres per minute, more suitably at least about 30 litres per minute. A relatively high flow rate of inert gas may advantageously improve the efficiency of extraction from the tobacco starting material. Optionally, the heating step may be carried out under vacuum. Suitable heating methods for carrying out the heating of the tobacco starting material are known to the skilled person and include: dry distillation, hydrodistillation, vacuum distillation, flash distillation and thin film hydrodistillation.

Where the volatile compounds are collected by absorption in a liquid solvent the step of forming the liquid tobacco extract can comprise drying the solution of the volatile compounds in the liquid solvent in order to concentrate the solution. Drying may be carried out using any suitable means, including but not limited to desiccation, molecular sieves, freeze drying, phase separation, distillation, membrane permeation, controlled crystallisation of water and filtering, reverse hygroscopicity, ultracentrifugation, liquid chromatography, reverse osmosis or chemical drying.

The liquid tobacco extract is particularly suitable for producing a composition or formulation or gel composition, for use in an aerosol-generating system. An aerosol-generating system comprising the composition or formulation or gel composition is disclosed. In such an aerosol-generating system, the composition or formulation or gel is typically heated within an aerosol-generating device—such as a device comprising a heater element that interacts with the composition or formulation or gel incorporating the liquid tobacco extract to produce an aerosol. During use, volatile compounds are released by heat transfer and entrained in air drawn through the aerosol generating device. As the released compounds cool they condense to form an aerosol that is inhaled by the consumer.

The invention is further described in the Examples below, which are provided to describe the invention in further detail. These examples, which set forth a preferred mode presently contemplated for carrying out the invention, are intended to illustrate and not to limit the invention.

### EXAMPLES

Example 1—Materials & Methods Plant material Prior to germination, the seeds are sterilized by a vapor chlorine gas method. A solution of 5% final chlorine is placed together with seed glass tubes in a bell jar. Hydrochloric acid (37%) is then added to the solution, and the seeds are incubated for 2 h. Then, under a laminar flow hood, the seeds are placed on Murashige & Skoog (Int J Mol Sci. (2020) 21(10):3441) growth medium and transferred to a plant growth room (24° C., 16 h light/20° C., 8 h dark) for 4 weeks. Well-developed plantlets are transferred to a greenhouse and cultivated in 10 L pots until fully grown (from 2 to 6 replicates). Artificial light is applied for 16 h every day. For each plant, representative fully grown leaves are sampled at flowering time. The collected samples are subsequently lyophilized and disrupted by shaking at 400 rpm in containers with glass beads: 8 h for leaves and 24 h for roots. Still undisrupted roots are at this point ground in a mortar to the finest level possible.

### Method for Determining Free Amino Acid, Sugar, Ammonia and Nitrate Levels

Amino acid content is measured using Method MP 1471 rev 5 2011, Resana, Italy: Chelab Silliker S.r.I, Merieux NutriSciences Company. For amino acid determination in cured plant leaves, after mid-rib removal, cured lamina is dried at 40° C. for 2-3 days, if required. Tobacco material is then ground in fine powder (−100 uM) before the analysis of amino acid content. Alternatively, amino acid content is measured in plant material as described in UNI EN ISO 13903:2005.

Reducing sugar content is measured using a segmented-flow colorimetric method developed for analysis of tobacco samples as adapted by Skalar Instrument Co (West Chester, PA) and described in Tobacco Science 20: 139-144 (1976). The measurement of reducing sugar content is also described in Coresta Recommended Method 38, CRM38, CRM and ISO 15154: 2003. For reducing sugar determination in cured leaves, after mid-rib removal, cured lamina is dried at 40° C. for 2-3 days, if required. Tobacco material is then ground in fine powder (−100 uM) before the analyses of reducing sugars. Alternatively, reducing sugar content is measured according to ISO 15154: 2003.

Nitrate content is measured using a Lachet QuikChem 8500 instrument in accordance with the manufacturer's protocol (Lachat QuikChem method 12-107-04-1-J, Lachet Instruments, Loveland, CO, USA). Alternatively, nitrate content is measured according to ISO 15517:2003. Ammonia content is measured using ion chromatography according to ISO 21045:2018.

### Gene Expression Analysis

Sequencing data generated is demultiplexed by Illumina BaseSpace® Clarity LIMS (© Illumina, Inc.) and subsequently imported to Qiagen CLC Genomics Workbench version 12.0.1 (CLC bio, a QIAGEN Company). Transcriptome reads are mapped to the updated version of the N. tabacum reference genome (BMC Genomics (2017) 18(1):448) by the ‘RNA-Seq Analysis’ 2.16 tool with similarity of 0.8 (S=0.8) and fraction length of 0.8 (L=0.8) as mapping criteria. Mismatch cost is set to 2, insertion cost to 3, and deletion cost to 3. Global alignment is not performed, whereas paired distances are detected automatically. Maximum number of read hits is set to 10, and paired reads are counted as one. Gene expression-FPKM values are retrieved for each gene in the reference genome and also for those without transcript models.

### RNAi Procedure

The specific DNA fragment (SEQ ID NO: 8) is selected for suppressing the expression of both NtLKR-S and NtLKR-7). It is cloned between the strong constitutive MMV promoter and the 3′ nos terminator sequence of the nopaline synthase gene of Agrobacterium tumefaciens (Plant Mol Biol (1999) 40(5):771-82). The Burley tobacco variety TN90 and the Virginia tobacco variety K326 are transformed using standard Agrobacterium-mediated transformation protocols (Methods Mol Biol. (2006) 343:143-54 and Transgenic Res. (2013) 22(3):643-9). Seeds are harvested from three independent TO lines exhibiting the strongest NtLKR silencing. T1 plants from the 10 lines are grown in the greenhouse and selected by PCR for presence of construct insertion in the genomic DNA, with the following primers (5′-3′): MMV-F (SEQ ID NO: 9) and IPMS2-R (SEQ ID NO: 10). To verify that the progeny displays efficient transcript suppression, RNA is isolated from transgenic plants of each independent transformation event and their corresponding control plants, and qPCR experiments are performed to assess LKR gene expression levels using the primers NtLKR-F1 (SEQ ID NO: 6) and NtLKR-R1 (SEQ ID NO: 7).

### Example 2—Metabolomic Analyses

Metabolomic analyses revealed that the level of Lys increased in senescing tobacco leaves (after 48 h of Burley yellowing leaf during the air-curing process), as well as the catabolic products resulting from endogenous LKR activity—such as saccharopine, 2-aminoadipate and 2-ketoadipate, and pipecolate. In addition, transcriptomic analyses revealed that LKR genes involved in Lys degradation were also strongly upregulated during the yellowing phase. These data suggested that Lys accumulates in the early stage of curing.

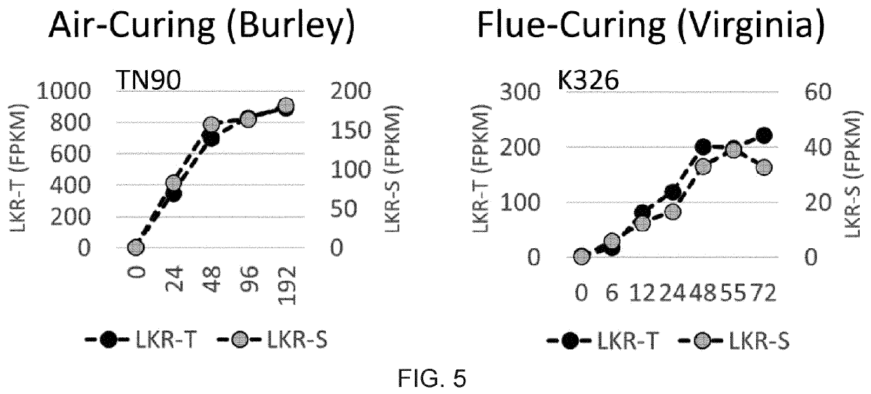

As shown in FIG. 5, NtLKR is strongly induced during the early curing phase, so-called yellowing (senescence genetic program, 0-192 h BU and 0-60 h FC).

### Example 3—Down-Regulation of NtLKR Via a RNAi Silencing Approach

NtLKR s down-regulated via a RNAi silencing approach to determine the chemical impact on cured leaf of the two tobacco types, Burley and Virginia. The growth of the plants is performed in the greenhouse. Classical agronomical practices are used for plant growing and curing (Industrial Crops and Products, 167, 2021, 113534, ISSN 0926-6690 and Leaf Curing Practices Alter Gene Expression and the Chemical Constituents of Tobacco Leaves. In: Ivanov, N. V., Sierro, N., Peitsch, M. C. (eds) The Tobacco Plant Genome. Compendium of Plant Genomes. Springer, Cham) such as air-curing for Burley and flue-curing for Virginia after harvesting the mature leaves.

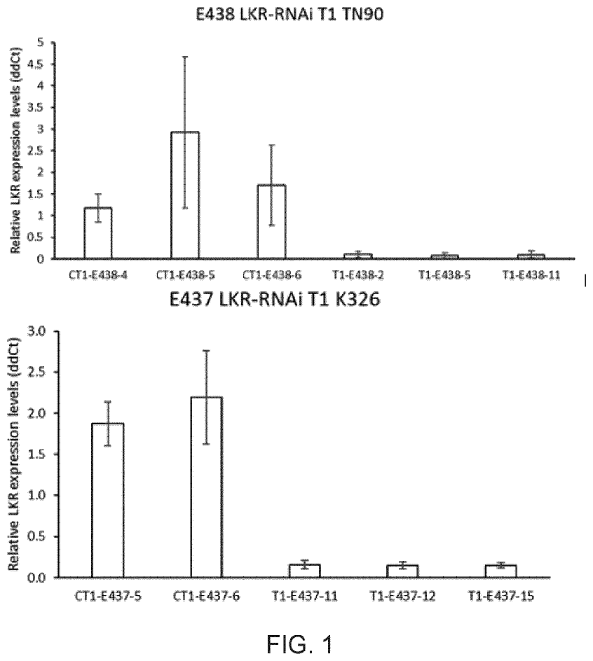

To investigate the function of the NtLKR genes and possible accumulation of Lys in cured leaves when NtLKR (LKR/SDH) is down-regulated, NtLKR-RNAi plants are generated using the insert of SEQ ID NO: 8 as RNAi-construct. No peculiar phenotypes are observed when growing anti-NtLKR T1 plants compared to wild-type plants. Several transformed lines are cultivated in TO, screened and selected via RT-qPCR (using full green midrib/lamina as tissues to isolate RNA). The seeds of the plants exhibiting a marked down-regulation of NtLKR are grown again (T1 plants) and rescreened by RT-qPCR to confirm the NtLKR silencing (see FIG. 1). Based on the gene expression levels, three NtLKR-T1 lines are selected in both tobacco types Burley TN90 and Virginia K326 for further analyses of free amino acids, sugars, ammonia and nitrate contents.

### Example 4—Impact of NtLKR Silenced Lines on Senescence in Burley TN90 Background

NtLKR-RNAi Burley TN90 plants are very efficient in accumulating more Lys and some other amino acids (Arg), thus the impact on yellowing (assuming that Lys is a source of energy for yellowing leaves) is tested in middle stalk leaf.

Control and T2-NtLKR-RNAi plant in Burley TN90 background are grown in a greenhouse in a randomized way. After 2 months of growing, all the plants are topped and half the culture is switched from Burley nutritive solution (EC=2.4) to water (EC=0.8). The other half of the culture remains with the Burley nutritive solution.

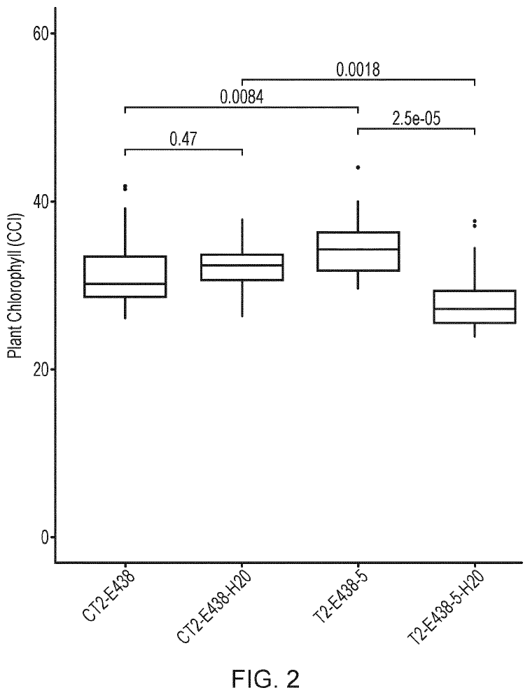

The data shows (see FIG. 2) the chlorophyll measures (CCI, 3 measures per plant) on C stalk position 21 days after nutritive solution switch.

Chlorophyll measurement (CCI) in middle stalk leaf position in control (CT2-E438) and LRK-RNAi (T2-E438-5) is shown in Table 1. Plants are topped after 2 months of growing. At that time, half of the culture is switched for water (H20, EC=0.8) instead of nutritive solution (EC=2.4). The chlorophyll (CCI) is recorded 21 days after. Numbers show t-test p values.

Interestingly, the absence of NtLKR gene (copies S and T) induced a faster chlorophyll decrease. This indicates an early senescence of those plants compare to control. Therefore, the accumulation of Lys contributes to a faster leaf senescence.

### Example 5—Impact of NtLKR Down-Regulation (T1 Plants) on Burley Tobacco TN90 Chemistry

The cured leaf free amino acids, sugars, ammonia and nitrate are determined in control (WT, n=14) and NtLKR-RNAi (n=13) plants. No differences are found for sugars, ammonia and nitrate for the Burley type. Table 2 depicts for fully cured tobacco the mean of the single amino acids in control and NtLKR-RNAi plants, as well as the percentage/fold change and statistical relevance (p-value ANOVA). As suspected by blocking the activity of NtLKR/SDH, Lys significantly increases in the cured leaf lamina by a factor of about 11×, thus changing the chemistry of final material compared to control leaves. The content of other amino acids is significantly altered, but to a lesser extent than Lys, namely: Arg, Pro, GABA, Gln, Leu, Ala, Phe, Tyr, lieu, Met, Thr and Gly (see Table 2). Interestingly, Met decreased by a factor of three. This attests to a strong rearrangement of the free amino acids in cured leaf compared to green leaves, mostly conducted by the activity of the aminotransferases during leaf yellowing (early curing), as shown in Bovet et al. (2019) Plants (Basel) 11; 8(11):492. In addition to asparagine synthetase, which is in fact an aminotransferase (WO2017042162), others play a role in the reorganization of amino acids during leaf yellowing, such as diaminopimelate aminotransferase (DAPAT) involved in both catabolism and anabolism of lysine, and aspartate amino transferases (AAT) also expressed during senescence and having the potential to change the leaf chemistry after curing (WO2019185703). As photosynthesis is no more active during leaf yellowing, the only way for the early senescing leaf is to remobilize N-resources by transferring the amino groups to enter into the energy machinery like mitochondria to produce reducing equivalent (NADPH or NADH) or to produce cytoplasmic Lys (see FIG. 2), and Asn (Bovet et al. (2019) Plants (Basel) 11; 8(11):492) for the N-storage in seeds. By blocking the NtLKR activity and accumulating Lys, this leads to change the balance of free amino acids in the cured leaf.

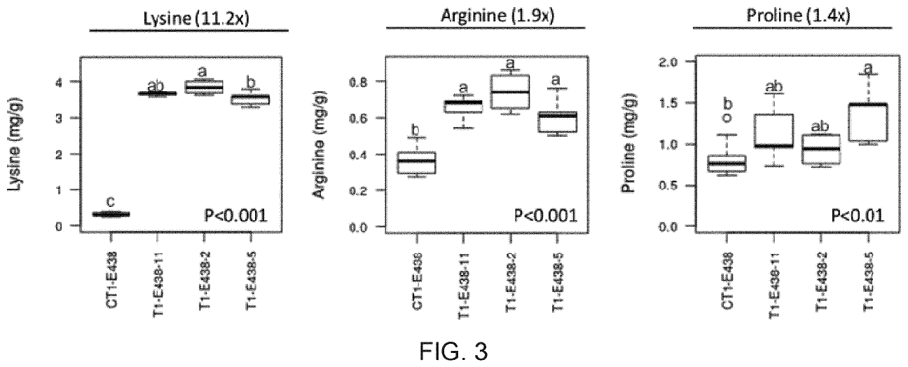

The increase of Lys (>11×) as shown in FIG. 3 is perfectly aligned with the silencing of NtLKR in the three different independent RNAi lines (see FIG. 1). This also confirms the activity of Nitab09g012060.1.1 and Nitab18g020540.1.1 as LKR enzymes (LKR-T and LKR-S, respectively). Although the content of Lys within the green leaves of NtLKR-RNAi lines is not measured, it is likely in frame of the expression profiles of NtLKR-S and NtLKR-T, functioning as Senescence-Activated Genes that the accumulation of Lys in cured leaves is associated with the senescence process. When the yellowing phase takes place naturally on the stalk, we can assume that Lys and Asn are then remobilized and transported to the seeds via the phloem. The increase of Arg and Pro, although minor, was also quite clear in the three independent lines, and resulted from NtLKR inactivation.

### Example 6—Impact of NtLKR Down-Regulation (T1 Plants) on Virginia Tobacco K326 Chemistry

Free amino acids, sugars, ammonia and nitrate are analysed in control (WT, n=8) and NtLKR-RNAi (n=13) cured leaf lamina of Virginia tobacco. In contrast to Burley, Virgina types needs less N-fertilization, and accumulates less free nitrate, ammonia and free amino acids, but more reducing sugars in cured leaves. Tables 3 and 4 depict for fully cured tobacco the mean of the free amino acids, sugars, ammonia and nitrate in both control and NtLKR-RNAi plants, as well as the percentage/fold change and statistical relevance (p-value, ANOVA). As suspected, by blocking the activity of NtLKR/SDH, Lys also significantly increased in Virginia, similarly to Burley cured leaves, but to a lesser extent, the factor being of about 4×.

Such an accumulation of Lys in Virginia (4×) is accompanied by significant changes in other free amino acids, reducing sugars, and nitrate. The content of the other amino acids subjected to changes, are: Arg, Gln, His, Tyr, Trp, Thr, GABA, Asn, Ala, lieu, Val and Ser (see Table 3). Considering the fact, that the total amino acid content in Virginia is about ten times less high compared to Burley (compare Table 2 and Table 3), the impact of silencing NtLKR genes on amino acid of cured Virginia leaves is weaker. However, a significant 20% decrease of glucose and fructose, and a 34% decrease of nitrate compared to WT is observed. Regarding nitrate, the values are so low, that it is difficult to conclude about a correlation with the lack of active NtLKR/SDH. On the other hand, the 20% reduction of each main reducing sugars is more consistent. Indeed, when both glucose and fructose are evaluated, it makes a total of reducing sugars corresponding to 17.04 in WT and 13.49 in NtLKR-RNAi, thus a decrease of about 3.5% of reducing sugars in the transgenic lines. No changes are observed in the ammonia content, like for Burley (for which this gene was initially targeted).

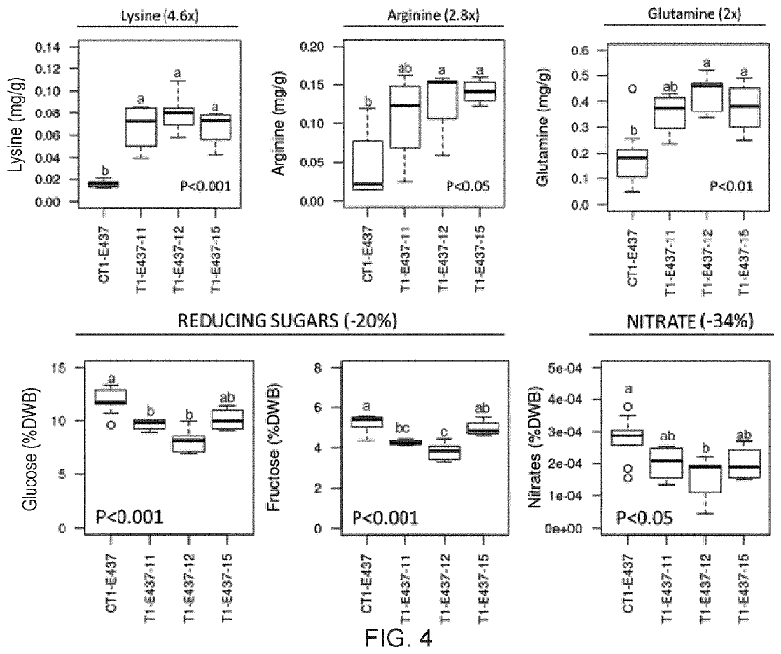

Data presented in FIG. 4 attests that the significant increase of Lys (>4×) is perfectly aligned with the silencing of NtLKR in the three different independent RNAi lines (see FIG. 2). This also confirms the activity of Nitab09g012060.1.1 and Nitab18g020540.1.1 as LKR enzymes (LKR-T and LKR-S, respectively). The increase of Arg and Gln, although minor, is also significant in the three independent lines and resulted from NtLKR inactivation. The global 20% decrease of glucose and fructose in the Virginia NtLKR-RNAi plants, even reached 30% in the line E437-12. Such a decrease may result either from more carbon respiration or assimilation of aa already within the green leaves or less accumulation of starch within the chloroplasts. It is clear, that a decreased NtLKR activity has low impact on the biological function of the vegetative plants, not impacting fitness and the growth of the transgenic plant, but it is exemplified once entering the senescence program.

We observed that when NtLKR is down-regulated, Lys increased whereas methionine and threonine decreased (particularly in the N-accumulating tobacco type Burley) and the decrease of glucose, as a reduced source of energy for the mitochondria, was rather found in the high reducing sugar accumulator Virginia.

### Example 7—Impact on Sensory in RNAi Modified Burley TN90 Plants

The altered sensory profile is presented in Table 5. The accumulation of Lys in modified Burley TN90 contributes to decreased harshness, makes the aerosol more round and smooth with less typical dark notes, but with more animalic and nutty notes (less trigeminals impact).

### Example 8—Impact on Sensory in RNAi Modified Virginia K326 Plants

The altered sensory profile is presented in Table 6 and contributes to increased hay notes with a rounder and darker flavour profile.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cellular, molecular and plant biology or related fields are intended to be within the scope of the following claims.

SEQUENCES
Polynucleotide coding sequence of LKR-S from Nicotiana tabacum
SEQ ID NO: 1
atgactgatttgctaaaatttggtcgagagattcttggtccagtcataatgtttggaaatggagtagttggtatc
ctctcagaggcgaccaacaaatgggagagacgagctcctctgactccttcacactgtgctcgattgctgcatggt
gggaggggtaagactggagtgtcgcgcatcatcatgcagccatccacgaagcgtgttcatcatgatgctctctat
gaggacgttggatgcgagatttctgaggacttgtccgactgtggtctcatattgggtatcaaacagcccaagttg
gagatgattcttcctgatagggcctatgcattcttctcgcatactcacaaggcacaaaaggagaatatgcccttg
ttggataagatattagctgaaagggcatctctgttcgactacgagcttatagttggagacactgggaagaggtta
cttgcatttggaagttttgctggtcgagctggaatgattgattttctacgcggattaggactgtggtatctcaac
catggctactcaacgccctttctctcactcggctcatcgtacatgtactcttcattggctgctgctaaagctgca
gtaatttctgttggcgaagagatagcgaccatggggcttccggcgggaatatgtccccttgtatttgttttcact
ggttctggaaacgtgtctcgtggtgcacaagaaatctttaagcttcttcctcatacttttgtggatccaagaaaa
ctttcagaactacatgagacagctagggatcttactcaatccaaacatccatcaaagagaatctttcaagtgtat
ggttgcgtaacgacctgccaagacatggtggaacatttgaatccttcaaaatcctttgataagattgattactat
gcacatccagagcaatacagacctgctttccatgagaaaatagccccttatgtatctgtcatagtaaattgtatg
tactgggagaagaggtttcctaggttgttgactactaagcagattcaagacctcatgagaaatggatgcccactt
gttggaatctgcgacataacatgtgatgtgggaggctcaatcgagttcatcaaccaaacatcatcaatagactcg
ccttttttcaggtacgaaccttccaatgattcatatcattatgacatagaaggtaaaggtgtgatgtgttcagct
gttgatattcttcccaccgagtttgcaaaagaggcatcccaacatttcggagatattttgtcccactttacggga
agcttagcttcctttagaaatcttgaggagttacctgcacacttaaagagagcttgcatagcccatcacggagct
cttactcagttgtatgaatacatccctcgaatgcggaaatctgacctagaagacccttcgacagttctttctagt
tccaatgcaaacgggaggaaatacactgttttggtatctctgagtggtcatttgtttgataaattcctcataaac
gaagctttggatattattgaagcagcaggtggctccttccacttggtgaagtgccaagttggtcaaatcacaagc
gctttgtcatactcagaactggaggttggagctgaagataaagcagttttagataaaatagttgattctttaact
tcccttgctaattcaagaaattctttgggatctcaaaacaaggaaaacaacatgatatcgttgaaggttggggaa
ttccaacaaagcatcatagatgagaaatctgatgcaaagaaggtcttaattttaggtgctggtcgggtttgcaga
ccagctgcagaacttttagcatcaataggtagcatgtcatccggacaatggttatcatctataactgctgatttt
gaagagcaacattgtgttcaagtaattgttgcttctttgtacttgaaggatgcagaagaggttactgaaggcatt
ccaaatgcaaaagcagttcaacttgatattatgaatcatgagtctctttctagttgcatctcacaggttgatgtt
gtcatcagcttactgcctcctagttgccatggtattgtagcaaaatcatgcattgagctgaagaaacatcttgtc
acagctagctacgttaatgattctatgttaaagctagacgaagacgcaaaatgtgctggaattactattcttggt
gaaatgggcttggacccaggaatagtaactctaataagagtgtcaatgaatttgttggaaaattcttcatggtat
gaaatttcctctgaatgtgattgtgcataa
Polypeptide sequence of LKR-S from Nicotiana tabacum
SEQ ID NO: 2
MTDLLKFGREILGPVIMFGNGVVGILSEATNKWERRAPLTPSHCARLLHGGRGKTGVSRIIMQPSTKRVHHDALY
EDVGCEISEDLSDCGLILGIKQPKLEMILPDRAYAFFSHTHKAQKENMPLLDKILAERASLFDYELIVGDTGKRL
LAFGSFAGRAGMIDFLRGLGLWYLNHGYSTPFLSLGSSYMYSSLAAAKAAVISVGEEIATMGLPAGICPLVFVFT
GSGNVSRGAQEIFKLLPHTFVDPRKLSELHETARDLTQSKHPSKRIFQVYGCVTTCQDMVEHLNPSKSFDKIDYY
AHPEQYRPAFHEKIAPYVSVIVNCMYWEKRFPRLLTTKQIQDLMRNGCPLVGICDITCDVGGSIEFINQTSSIDS
PFFRYEPSNDSYHYDIEGKGVMCSAVDILPTEFAKEASQHFGDILSHFTGSLASERNLEELPAHLKRACIAHHGA
LTQLYEYIPRMRKSDLEDPSTVLSSSNANGRKYTVLVSLSGHLFDKFLINEALDIIEAAGGSFHLVKCQVGQITS
ALSYSELEVGAEDKAVLDKIVDSLTSLANSRNSLGSQNKENNMISLKVGEFQQSIIDEKSDAKKVLILGAGRVCR
PAAELLASIGSMSSGQWLSSITADFEEQHCVQVIVASLYLKDAEEVTEGIPNAKAVQLDIMNHESLSSCISQVDV
VISLLPPSCHGIVAKSCIELKKHLVTASYVNDSMLKLDEDAKCAGITILGEMGLDPGIVTLIRVSMNLLENSSWY
EISSECDCA
Polynucleotide coding sequence of LKR-T from Nicotiana tabacum
SEQ ID NO: 3
atgtttggaaatggtgtagttggtatcctctcagaggcgacgaacaagtgggaaagacgagctcctctgactcct
tcacattgtgctcgattgctgcatactggagtgtcgcgcatcatcgtgcagccatccacaaagcgtgttcatcat
gatgctctctatgaggatgttggatgcgagatttctgaggacttgtccgactgtggtctcatattgggtatcaaa
cagcccaagttggagatgattcttcctgatagggcctatgcattcttctcacatactcacaaggcacaaaaggaa
aatatgcccttgttggatatgatactagctgaaagggcttctctgttcgactatgagcttatagttggagacact
gggaagaggttacttgcgtttggaagttttgctggtcgagctggaatgattgattttctacgcggattaggactg
tggtatctcaaccatggctactcaacgccctttctctcactcggctcatcgtacatgtactcttcattggctgct
gctaaggctgcagtaatttctgttggcgaagagatagcgaccatggggcttccagcgggaatatgtcctcttgta
tttgttttcactggtgctggaaatgtgtctcgtggtgcacaagaaatttttaagcttcttcctcatacatttgtg
gatccaagaaaactttcagaactacatgagacggctagggatcttactcaatccaaacatccatcaaagagaatc
tttcaagtgtatggttgcgtaacgacctgccaagacatggtggaacgtttaaatccttcaaaatcctttgataag
attgattactatgcacatccagagcaatacggacctgctttccatgagaaaatagccccttatgcatctgtcata
gtaaattgtatgtactgggagaagaggtttcctaggttgttgactactaagcagattcaagacctcatgagaaat
ggatgcccacttgttggaatctgtgacataacatgtgatgtgggaggctcgatcgaattcatcaaccaaacatca
tcaatagactcgccttttttcaggtatgaaccttccaatgattcatatcattttgacatagaaggtaaaggtgtg
atgtgttccgctgttgatattcttcccaccgagtttgcaaaagaggcatcccaacatttcggagatattttgtcc
cactttacgggaagcttagcttcctttagaaatcttgaggagagagcttgcatagcccatcacggacgtcttact
cagttgtatgaatacatccctcgaatgcggaaatctgacctagaagacccttcaacagttctttctagttccaat
gcaaacgggaggaaatacaccgttttggtatctctgagtggtcatttgtttgataaattcctcataaacgaagcc
ttggatattattgaagcagcaggtggctccttccacttggtgaagtgccaagttggtcaaatcacaagcgctttg
tcatactcagaactggaggttggagctgaagataaagcagttttggataaaatagttgattctttaacttccctt
gcttattcaagaaattctttgggatctcaaaacaaggaaaacaacatgatatctttgaaggttggggaattccaa
caaagcatcacagatgagaaatctgatgcaaaggtcttaattttaggtgctggtcgggtttgcagaccagctgca
gaacttttagcatcaataggtagcatgtcatccggacaatgcttatcatctataactgctgattttgaagagcaa
aattgtgttcaagtgattgttgcttctttgtacttgaaggatgcggaagaggttactgaaggtattccaaatgca
aaagcagttcagcttgatattacgaatcacgagtctctttctagttgcatctcacaggttgatgttgtcatcagc
ttactgcctcctagttgccatggtattgtagcaaaatcatgcattgagctgaagaaacatcttgtcacagctagc
tacgttaatgattcaatgttaaagctagacgaagacgcaaaatgtgctggaattactattcttggtgaaatgggc
ttggatccaggaatagatcatatgatggcaatgaaaatgatcaaccaagcccatgcagcaaacggaaagatcagg
tctttcgtttcttactgtggtggtcttccctctccagctgcggccaacaacccactagcttataagttcagttgg
aacccagccggagctatacgagctgggtggaatccagcagcctatagatctgaaggggaaattattcatgttgaa
ggtcagaggctttatgattcagctgcaaagcttcgtcttcctgattttccagcttttgcattagagtgtctccca
aatcgcaactccttagtgtatggagacttatatggtgtaggagaggaagcatcaacaatctttcgaggaacacta
cgctatgaaggttttagtcaaataatggggacacttgccaagattggattcttctgtacagaaccatctcttatt
cttaaagatgggattaaacccacacacagagcatttttgctgggacttgttggaatagacggacagattttcccc
gaaccagtaattgatgaaaaatacatcactgataggatcttgaaacttgggctttgtaaagacaaggacactgca
gttaagacagcaaaaactataatatttttgggatttcaagagcctacagaaataccatcatcctgcaaatctcca
tttgaagttacgtgcttgcgcatggaagagagattagcatactccaatacagagcaggacatggtgcttttacac
catgaagtggtagtagattacccagatggccatgctgaaacccatagatccacacttttggaaatggggaggaca
gcgaatgggaaaaccaacatggccatgtctcttacagtagggattcccgcagccactggagctctgctattactt
gcgaacaagatcaaagcaaatggtgtactaaggcctattgatccagaagtttatgagccagcactggatattttg
gaggcatatggtttcaagttgctggagaagattgaataa
Polypeptide sequence of LKR-T from Nicotiana tabacum
SEQ ID NO: 4
MFGNGVVGILSEATNKWERRAPLTPSHCARLLHTGVSRIIVQPSTKRVHHDALYEDVGCEISEDLSDCGLILGIK
QPKLEMILPDRAYAFFSHTHKAQKENMPLLDMILAERASLFDYELIVGDTGKRLLAFGSFAGRAGMIDFLRGLGL
WYLNHGYSTPFLSLGSSYMYSSLAAAKAAVISVGEEIATMGLPAGICPLVFVFTGAGNVSRGAQEIFKLLPHTFV
DPRKLSELHETARDLTQSKHPSKRIFQVYGCVTTCQDMVERLNPSKSFDKIDYYAHPEQYGPAFHEKIAPYASVI
VNCMYWEKRFPRLLTTKQIQDLMRNGCPLVGICDITCDVGGSIEFINQTSSIDSPFFRYEPSNDSYHFDIEGKGV
MCSAVDILPTEFAKEASQHFGDILSHFTGSLASERNLEERACIAHHGRLTQLYEYIPRMRKSDLEDPSTVLSSSN
ANGRKYTVLVSLSGHLEDKFLINEALDIIEAAGGSFHLVKCQVGQITSALSYSELEVGAEDKAVLDKIVDSLTSL
AYSRNSLGSQNKENNMISLKVGEFQQSITDEKSDAKVLILGAGRVCRPAAELLASIGSMSSGQCLSSITADFEEQ
NCVQVIVASLYLKDAEEVTEGIPNAKAVQLDITNHESLSSCISQVDVVISLLPPSCHGIVAKSCIELKKHLVTAS
YVNDSMLKLDEDAKCAGITILGEMGLDPGIDHMMAMKMINQAHAANGKIRSFVSYCGGLPSPAAANNPLAYKFSW
NPAGAIRAGWNPAAYRSEGEIIHVEGQRLYDSAAKLRLPDFPAFALECLPNRNSLVYGDLYGVGEEASTIFRGTL
RYEGFSQIMGTLAKIGFFCTEPSLILKDGIKPTHRAFLLGLVGIDGQIFPEPVIDEKYITDRILKLGLCKDKDTA
VKTAKTIIFLGFQEPTEIPSSCKSPFEVTCLRMEERLAYSNTEQDMVLLHHEVVVDYPDGHAETHRSTLLEMGRT
ANGKTNMAMSLTVGIPAATGALLLLANKIKANGVLRPIDPEVYEPALDILEAYGFKLLEKIE
NtLKR-RNAi insert:
SEQ ID NO: 5
caggttgatgttgtcatcagcttactgcctcctagttgccatggtattgtagcaaaatcatgcattgagctgaag
aaacatcttgtcacagctagctacgttaatgattc
NtLKR-F1 primer for qPCR
SEQ ID NO: 6
atattattgaagcagcaggtggc
NtLKR-R1 primer for qPCR
SEQ ID NO: 7
tgctttatcttcagctccaacct
DNA fragment used for suppressing the expression of NtLKR-S and NtLKR-T
SEQ ID NO: 8
Caggttgatgttgtcatcagcttactgcctcctagttgccatggtattgtagcaaaatcatgcattgagctgaag
aaacatcttgtcacagctagctacgttaatgattc
MMV-F primer
SEQ ID NO: 9
gacgtctaatcccaacttcgtc
IPMS2-R primer
SEQ ID NO: 10
gacgtctaatcccaacttcgtc

TABLE 1
Chlorophyll measurement (CCI) in middle stalk leaf position
in control (CT2-E438) and LRK-RNAi (T2-E438-5)

| Plant ID | Plant n= | Mean (CCI) | SD |
|---|---|---|---|
| CT2-E438 | 10 | 31.54 | ±4.32 |
| CT2-E438-H20 | 10 | 32.23 | ±2.80 |
| T2-E438-5 | 7 | 37.79 | ±3.81 |
| T2-E438-5-H20 | 7 | 28.40 | ±4.15 |

TABLE 2
Free amino acids content in control and LKR-RNAi Burley plants (average of data in single
lines (WT n = 14 and LKR-RNAi, n = 13) as well the percentage/fold change and statistical
relevance (p-value). Statistical analyses performed with ANOVA, Tukey's HSD test. ‘LKR-
RNAi’ results are for fully cured Burley leaf.

| Compounds | Control (mg/g) | LKR-RNAi (mg/g) | Percentage/fold change | p-value |
|---|---|---|---|---|
| Lysine | 0.32 | 3.65 | X11.20 | <2e−16 |
| Arginine | 0.35 | 0.66 | X1.85 | 4.35E−09 |
| Proline | 0.81 | 1.15 | X1.41 | 0.00368 |
| GABA | 0.73 | 0.92 | X1.26 | 0.0214 |
| Glutamine | 2.15 | 2.71 | X1.25 | 0.0165 |
| Leucine | 0.17 | 0.21 | X1.24 | 0.000111 |
| Alanine | 2.02 | 2.44 | X1.20 | 0.00125 |
| Phenylalanine | 2.64 | 3.06 | X1.15 | 0.0203 |
| Tyrosine | 0.338 | 0.368 | X1.09 | 0.0135 |
| Isoleucine | 0.086 | 0.092 | X1.06 | 0.0277 |
| Methionine | 0.036 | 0.025 | −29% | 2.79E−05 |
| Threonine | 0.13 | 0.11 | −10% | 0.00249 |
| Glycine | 0.167 | 0.155 | −8% | 0.00249 |
| Asparagine | 35.57 | 32.5 | NA | 0.08 |
| Aspartic acid | 3.12 | 2.93 | NA | 0.707 |
| Tryptophan | 2.01 | 2.12 | NA | 0.0843 |
| Histidine | 1.77 | 1.72 | NA | 0.649 |
| Glutamic acid | 0.35 | 0.38 | NA | 0.679 |
| Serine | 0.35 | 0.32 | NA | 0.0706 |
| Valine | 0.124 | 0.121 | NA | 0.17 |
| Total free amino acids | 53.34 | 55.75 | NA | 0.418 |

TABLE 3
Free amino acid content in control and LKR-RNAi Virginia plants (average of data in single
lines (WT n = 8 and LKR-RNAi, n = 13), as well the percentage/fold change and statistical
relevance (p-value, ANOVA Tukey's HSD test). ‘LKR-RNAi’ results are for fully cured
Virginia leaf.

| Compounds | Control (mg/g) | LKR-RNAi (mg/g) | Percentage/fold change | p-value |
|---|---|---|---|---|
| Lysine | 0.0157 | 0.07 | X4.6 | 8.39E−06 |
| Arginine | 0.04 | 0.125 | X2.81 | 0.0126 |
| Glutamine | 0.189 | 0.39 | X2.06 | 0.00219 |
| Histidine | 0.04 | 0.07 | X1.68 | 0.00676 |
| Tyrosine | 0.07 | 0.124 | X1.62 | 0.00687 |
| Tryptophan | 0.11 | 0.18 | X1.6 | 0.00421 |
| Threonine | 0.05 | 0.09 | X1.53 | 0.00528 |
| GABA | 0.13 | 0.19 | X1.45 | 0.00399 |
| Asparagine | 0.16 | 0.23 | X1.38 | 0.0306 |
| Alanine | 0.2 | 0.27 | X1.30 | 0.00102 |
| Isoleucine | 0.063 | 0.038 | −39% | 0.0148 |
| Valine | 0.43 | 0.36 | −17% | 0.0105 |
| Serine | 0.11 | 0.08 | −15% | 0.00682 |
| Proline | 1.18 | 0.99 | NA | 0.33 |
| Aspartic acid | 0.43 | 0.57 | NA | 0.224 |
| Leucine | 0.09 | 0.07 | NA | 0.123 |
| Phenylalanine | 0.088 | 0.119 | NA | 0.136 |
| Glutamic acid | 0.06 | 0.07 | NA | 0.069 |
| Methionine | 0.013 | 0.015 | NA | 0.125 |
| Total free amino acids | 3.48 | 4.13 | NA | 0.245 |

TABLE 4
Sugar, ammonia and nitrate content in control and LKR-RNAi Virginia plants (average of
data in single lines (WT n = 8 and LKR-RNAi, n = 13), as well the percentage/fold change and
statistical relevance (p-value, ANOVA Tukey's HSD test). ‘LKR-RNAi’ results are for fully
cured Virginia leaf.

| Compounds | Control (mg/g) | LKR-RNAi (mg/g) | Percentage/fold change | p-value |
|---|---|---|---|---|
| Glucose | 11.85 | 9.2 | −23% | 9.08E−05 |
| Fructose | 5.19 | 4.29 | −18% | 2.85E−05 |
| Sucrose | 12.02 | 11.3 | NA | 0.216 |
| Sum of sugars | 29.07 | 24.8 | −15% | 2.45E−05 |
| Ammonia | 0.0061 | 0.007 | NA | 0.172 |
| Nitrates | 2.80E−04 | 1.80E−04 | −34% | 0.021 |

TABLE 5
Sensory analysis of RNAi modified Burley tobacco variety TN90 plants

| | 60% LKR(TN90)/40% FC BR | |
|---|---|---|
| | Control LKR TN90 60/40 | T1 LKR-RNAI 60/40 |
| Panelist 1 | Some darkness, slightly greenish, | Less dark and more animalic, spicy, |
| | some harshness, FC perceived at the | peperry notes than CT, less mushroom- |
| | blended side, a bit dusty, earthy- | earthy notes, some nutty notes perceived, |
| | mushroom notes, tingling the | loose the strong dark character, more |
| | mouth, coating and some dryness, | round, salivating |
| | some off-notes (soapy and greenish) | |
| Panelist 2 | Dark, earthy notes, good response on | Reduced darkness, more animalic and less |
| | mouth and throat, some dusty | mushroom notes, more clean and round, |
| | perception on aftertaste, a bit dry, | clearly less trigeminals (on mouth and |
| | mushroom notes | throat), more smooth, less off-notes on |
| | | aftertaste. |
| Panelist 3 | Cocoa, earthy, toasted, somehow | Perfumy, clearly rounder than CT, Brazil |
| | leathery, quite clean, some nutty | nut aroma, slight impact, smoother than |
| | notes, tingling the tongues, some | control, hashy at the end |
| | impact, harshness in throat | |
| Summary | Classical TN90 dark notes perceived, | Reduced harshness, more round and |
| | earthy-mushroom, some strength | smooth, less typical darkness and more |
| | and impact in throat | animalic and nutty notes, less trigeminals |

TABLE 6
Sensory analysis of RNAi modified Virginia tobacco variety K326 plants

| | 60% LKR(K326)/40% FC BR | |
|---|---|---|
| | Control CT-K326 GH | T1 LKR-RNAI |
| Panelist 1 | Smooth, salivatory, some sweet notes, | More perception of the hay notes, less |
| | some hint of green tobacco notes, very | greenish acidic notes, a bit less sweet, |
| | smooth in throat and chest, some hay | no changes in trigeminals, a bit more |
| | notes, a bit of coating with sweet-sour | dusty-earthy, moving the profile |
| | like notes, easy going | towards a bit more dark, rounder |
| Panelist 2 | Floral, bright, somehow perfumy, low | Toasted, nutty, strong hay notes, less |
| | aroma intensity, hay notes, bread, acidic | clean (dusty), some leathery notes, |
| | stream, smooth with slight impact on the | sweet. Rounder than the control, |
| | chest, some salivation | somewhat darker than the control, |
| | | slightly more powerful than the control, |
| | | still acidic, some salivation and coating |
| Panelist 3 | Some fishy notes at start (1-2 first puffs, | No off-notes at start, a bit more spicy |
| | nutty-hay, metallic notes, a bit of agrume | and aromatic hay notes, more |
| | peel notes | consistent puff-by-puff, more |
| | (acidity), sweet, some | salivation, more fullness in mouth, |
| | greenish unmature notes, on the bright | some harshness and darkness impacting |
| | side, easy to smoke, very low harshness | the throat, more smoky-ashy than the |
| | and tingling | control, more blended sensation, still |
| | | acidic, sweet and nutty, more aftertaste |
| | | (smoky) and salivation. |
| Summary | Weak trigeminal sensation, classical hay- | Rounder than the control, more hay |
| | nutty notes, sweet and low in aroma | notes, more dark, a bit less sweet but |
| | | still acidic, more trigeminal sensation |
| | | and fullness; smoky, earthy and |
| | | leathery notes perceived |

1. A mutant, non-naturally occurring or transgenic Nicotiana tabacum plant or part thereof having modulated expression or activity of lysine ketoglutarate reductase (LKR), said LKR comprising, consisting or consisting essentially of:
(i) a polynucleotide(s) comprising, consisting or consisting essentially of a sequence having at least 88% sequence identity to the SEQ ID NO: 1 (NtLKR-S) and/or having at least 86% sequence identity to SEQ ID NO: 3 (NtLKR-T);
(ii) a polypeptide(s) encoded by the polynucleotide set forth in (i);
(iii) a polypeptide comprising, consisting or consisting essentially of a sequence having at least 89% sequence identity to SEQ ID NO: 2 (NtLKR-S) and/or having at least 88% sequence identity to SEQ ID NO: 4 (NtLKR-T); or
(iv) a construct, vector or expression vector comprising the isolated polynucleotide(s) set forth in (i),
wherein said plant or part thereof comprises at least one modification which is capable of modulating: (a) the expression of the polynucleotide(s) in the plant or part thereof; or (b) the activity of the polypeptide(s) in the plant or part thereof, as compared to a control plant or part thereof in which the expression of the polynucleotide(s) or the activity of the polypeptide(s) has not been modified.

2. The mutant, non-naturally occurring or transgenic Nicotiana tabacum plant or part thereof according to claim 1, wherein the modification comprises at least one genetic alteration in a coding sequence of the polynucleotide(s) or in a regulatory region of the polynucleotide(s); and/or
wherein the modification comprises one or more of exogenous DNA or exogenous RNA; and/or
wherein the modification comprises one or more of a vector or a viral vector or an Agrobacterium vector or a CRISPR vector; and/or
wherein the modification is capable of driving one or more of RNA interference or transcriptional gene silencing or virus induced gene silencing; and/or
wherein the modification is capable of expressing one or more of double stranded RNA (dsRNA) or hairpin RNA (hpRNA) or small interfering RNA.

3. The mutant, non-naturally occurring or transgenic Nicotiana tabacum plant or part thereof according to claim 1, wherein the modulated expression or activity of LKR confers a modulation in the level of one or more amino acids in the plant or part thereof as compared to the level of the one or more amino acids in the control plant, suitably, wherein the modulated expression or activity of LKR confers a modulation in the timing of leaf senescence; suitably,
wherein the amino acid is lysine.

4. The mutant, non-naturally occurring or transgenic Nicotiana tabacum plant or part thereof according to claim 1, wherein the part of the mutant, non-naturally occurring or transgenic Nicotiana tabacum plant is cured or dried leaf; suitably,
wherein the levels of at least lysine, arginine, GABA, glutamine, alanine, tyrosine, isoleucine and threonine are modulated in the cured or dried leaf as compared to cured or dried leaf from the control plant.

5. The mutant, non-naturally occurring or transgenic Nicotiana tabacum plant or part thereof according to claim 1, wherein the Nicotiana tabacum plant or part thereof is a Burley type; suitably,
wherein in the cured or dried leaf the levels of at least lysine, arginine, proline, GABA, glutamine, leucine, alanine, phenylalanine, tyrosine, isoleucine, methionine, threonine and glycine are modulated and there is no significant change in the levels of at least asparagine, aspartic acid, tryptophan, histidine, glutamic acid, serine, and valine as compared to cured or dried leaf from the control plant; and/or
wherein in the cured or dried leaf the expression of the LKR polynucleotide or the activity of the LKR polypeptide is decreased or inhibited and wherein in the cured or dried leaf: (i) the levels of at least lysine, arginine, proline, GABA, glutamine, leucine, alanine, phenylalanine, tyrosine, and isoleucine are increased; and (ii) the levels of at least methionine, threonine and glycine are decreased; and (iii) there is no significant change in the levels of at least asparagine, aspartic acid, tryptophan, histidine, glutamic acid, serine and valine compared to cured or dried leaf from the control plant.

6. The mutant, non-naturally occurring or transgenic Nicotiana tabacum plant or part thereof according to claim 1, wherein the Nicotiana tabacum plant or part thereof is a Virginia type; suitably,
wherein in the cured or dried leaf the levels of at least lysine, arginine, glutamine, histidine, tyrosine, tryptophan, threonine, GABA, asparagine, alanine, isoleucine, valine and serine are modulated and there is no significant change in the levels of at least proline, aspartic acid, leucine, phenylalanine, glutamic acid and methionine as compared to cured or dried leaf from the control plant.

7. The mutant, non-naturally occurring or transgenic Nicotiana tabacum plant or part thereof according to claim 6, wherein in the cured or dried leaf the expression of the LKR polynucleotide or the activity of the LKR polypeptide is decreased or inhibited and wherein in the cured or dried leaf: (i) the levels of at least lysine, arginine, glutamine, histidine, tyrosine, tryptophan, threonine, GABA, asparagine and alanine are increased; and (ii) the levels of at least isoleucine, valine and serine are decreased; and (iii) there is no significant change in the levels of at least proline, aspartic acid, leucine, phenylalanine, glutamic acid and methionine as compared to cured or dried leaf from the control plant.

8. The mutant, non-naturally occurring or transgenic Nicotiana tabacum plant or part thereof according to claim 6, wherein the sum of sugars is modulated; suitably,
wherein the sum of sugars is decreased.

9. Nicotiana tabacum plant material, cured Nicotiana tabacum plant material, or homogenized Nicotiana tabacum plant material, derived or obtained from the Nicotiana tabacum plant or part thereof of any of claim 1; suitably,
wherein the Nicotiana tabacum plant material is selected from the group consisting of biomass, seed, stem, flower, or leaf or a combination of two or more thereof; suitably,
wherein the Nicotiana tabacum plant material is leaf, suitably,
wherein the leaf is cured leaf; suitably,
wherein the cured leaf is selected from the group consisting of flue-cured leaf, sun-cured leaf or air-cured leaf.

10. A method for producing a Nicotiana tabacum plant in which the level of at least one amino acid is modulated comprising:
(a) providing a Nicotiana tabacum plant comprising:
(i) a polynucleotide(s) comprising, consisting or consisting essentially of a sequence having at least 88% sequence identity to the SEQ ID NO: 1 (NtLKR-S) and/or having at least 86% sequence identity to SEQ ID NO: 3 (NtLKR-T);
(ii) a polypeptide(s) encoded by the polynucleotide(s) set forth in (i);
(iii) a polypeptide(s) comprising, consisting or consisting essentially of a sequence having at least 89% sequence identity to SEQ ID NO: 2 (NtLKR-S) and/or having at least 88% sequence identity to SEQ ID NO: 4 (NtLKR-T); or
(iv) a construct, vector or expression vector comprising the isolated polynucleotide(s) set forth in (i); and
(b) introducing at least one modification which is capable of modulating: (a) the expression of the LKR polynucleotide(s) in the Nicotiana tabacum plant; or (b) the activity of the LKR polypeptide(s) in the Nicotiana tabacum plant, as compared to a control in which the expression of the LKR polynucleotide(s) or the activity of the LKR polypeptide(s) has not been modified.

11. The method according to claim 10, wherein in step (b) the at least one modification is introduced by genome editing; suitably,
wherein the genome editing is selected from CRISPR-mediated genome editing, mutagenesis, zinc finger nuclease-mediated mutagenesis, chemical or radiation mutagenesis, homologous recombination, oligonucleotide-directed mutagenesis and meganuclease-mediated mutagenesis; or
wherein in step (b) the at least one modification is introduced using an interference polynucleotide.

12. A Nicotiana tabacum plant obtained or obtainable by the method of claim 10.

13. A method of producing cured Nicotiana tabacum plant material having altered levels of at least one amino acid, comprising:
(a) producing a Nicotiana tabacum plant according to claim 10;
(b) harvesting plant material (for example, leaf) from the Nicotiana tabacum plant; and
(c) curing the plant material.

14. Cured Nicotiana tabacum plant material (for example, leaf) obtained or obtainable by the method of claim 13.

15. A tobacco product comprising the Nicotiana tabacum plant material, the cured Nicotiana tabacum plant material, or the homogenised Nicotiana tabacum plant material according to claim 9; suitably,
wherein the tobacco product is a tobacco blend; suitably,
wherein the tobacco blend comprises Virginia type tobacco and/or Burley type tobacco.
